SEQUENCE LISTING (9 sequences)

SEQ ID NO: 1 - DNA, 2170 bp, Aspergillus niger
gccattctttctctacaatctggtcccacttctttcgttcactctttcatttcgtgcctagcccgtatccttccttttagcgtaaccggctatctcccagcattcgacagtcaccttatcgtatctccaagtcgggccccgcaattgctcggcaggcacatgtacgcaacccttgatcattaagataattccaatctcccagtgactgtagccatggtctcaatgtcggccctgcggcacgggcttggggtcctctatcttgctagctggctggggtcgtcgctggctgccagcaccgagcaatggaagtcccggtcaatctatcagaccatgacggatcggttcgcgcgcactgatggctcaaccacctccccctgcaacaccacggagggtctgtactgtggtggtacctggcgcggcatgatcaaccatctggattacatccaggggatgggctttgatgccgtcatgatctcccctatcatcgagaacgtcgaaggtcgcgtggagtacggagaagcctatcacggctattggcccgtggatctgtactccctcaactcgcactttggaacccaccaggatctactggacctgagtgatgccctgcatgctcgcgacatgtatctgatgatggacacagtcatcaacaacatggcctacatcaccaatggctccgaccccgccacccacatcgactactcaaccctgacccccttcaatagctcgtcttactaccatccctactgtaagatcaccgactggaataacttcaccaatgcccagctgtgccaaaccggtgacaacatcgtggccttgcccgatctgtataccgagcatgccgaggtgcaagaaaccctgagcaactgggccaaggaagtcatctccacctattccatcgacggcctccgcatcgatgccgcaaaacatgtgaaccccggcttcctgaagaatttcggcgatgcgcttgatatcttcatgaccggcgaagtgctgcagcaagaggtcagcacaatctgcgattatcagaacaactacatcggcagtctcccgaattaccccgtctactacgccatgttgaaggcctttaccttgggcaacaccagcgccctggccactcaggtccagtcgatgaagaattcctgtaatgatgtgaccgccttgtcgtcattctccgaaaaccacgatgtcgcacgatttgccagcatgacccatgacatggctgtatgaactctgcttgactttacagacccctttcctagctaacattgtccctactagctcgcgaagaatattctcacattcactcttctcttcgacggtgttcccatgatctatcaaggtcaagagcagcatctcgacggccctggcagtccggaaaaccgagaagcgatatggctctctgagtacaacaccgacgccgagctctacaagttgatcggcaagttgaacgccatccggaagcacgcctaccgactcgacaatcactaccccgatgtcgagacgtaccccatcttcgaaggtggcagtgaactgggattccgcaagggaatcgaaggtcgacaggtggtcatgcttctgtccacgcaaggcacgaacagcagcgcttacaacctctcgatgccggtgagtttcacgggtggcacagtagtgaccgagattctgaactgcgtcaactacacggtcaacacccagagcgaactcgtggttccgatggataaaggagagccgcgcgtgttcttcccggcggatctgatgcccggcagtgggctgtgcggacttcctgtcgcgaatgtcacctatgctgccttgaggacgcagggtgcagcggcggcggaggctgccttgtcgctaggtatcaagactgatgcagcttctagtgctttgctttctctggggctgtctgtggtggcgggtctgattgtggggatgtggtaatatgctgatagctacagatgggttgagatatattatcatgtttcagtgttctttgtatgtatttgcagtaacgatacccttacctagtatattgtatatattcacttcgctatatccactactactcgcagatactagccgcatctcgctttattccacactagttactagcacttcagcactaagcaataaacagaacgaagacaaattactagtccagtatatatataaa

SEQ ID NO: 2 - DNA, 1668 bp, Aspergillus niger
atggtctcaatgtcggccctgcggcacgggcttggggtcctctatcttgctagctggctggggtcgtcgctggctgccagcaccgagcaatggaagtcccggtcaatctatcagaccatgacggatcggttcgcgcgcactgatggctcaaccacctccccctgcaacaccacggagggtctgtactgtggtggtacctggcgcggcatgatcaaccatctggattacatccaggggatgggctttgatgccgtcatgatctcccctatcatcgagaacgtcgaaggtcgcgtggagtacggagaagcctatcacggctattggcccgtggatctgtactccctcaactcgcactttggaacccaccaggatctactggacctgagtgatgccctgcatgctcgcgacatgtatctgatgatggacacagtcatcaacaacatggcctacatcaccaatggctccgaccccgccacccacatcgactactcaaccctgacccccttcaatagctcgtcttactaccatccctactgtaagatcaccgactggaataacttcaccaatgcccagctgtgccaaaccggtgacaacatcgtggccttgcccgatctgtataccgagcatgccgaggtgcaagaaaccctgagcaactgggccaaggaagtcatctccacctattccatcgacggcctccgcatcgatgccgcaaaacatgtgaaccccggcttcctgaagaatttcggcgatgcgcttgatatcttcatgaccggcgaagtgctgcagcaagaggtcagcacaatctgcgattatcagaacaactacatcggcagtctcccgaattaccccgtctactacgccatgttgaaggcctttaccttgggcaacaccagcgccctggccactcaggtccagtcgatgaagaattcctgtaatgatgtgaccgccttgtcgtcattctccgaaaaccacgatgtcgcacgatttgccagcatgacccatgacatggctctcgcgaagaatattctcacattcactcttctcttcgacggtgttcccatgatctatcaaggtcaagagcagcatctcgacggccctggcagtccggaaaaccgagaagcgatatggctctctgagtacaacaccgacgccgagctctacaagttgatcggcaagttgaacgccatccggaagcacgcctaccgactcgacaatcactaccccgatgtcgagacgtaccccatcttcgaaggtggcagtgaactgggattccgcaagggaatcgaaggtcgacaggtggtcatgcttctgtccacgcaaggcacgaacagcagcgcttacaacctctcgatgccggtgagtttcacgggtggcacagtagtgaccgagattctgaactgcgtcaactacacggtcaacacccagagcgaactcgtggttccgatggataaaggagagccgcgcgtgttcttcccggcggatctgatgcccggcagtgggctgtgcggacttcctgtcgcgaatgtcacctatgctgccttgaggacgcagggtgcagcggcggcggaggctgccttgtcgctaggtatcaagactgatgcagcttctagtgctttgctttctctggggctgtctgtggtggcgggtctgattgtggggatgtggtaa

SEQ ID NO: 3 - protein, 555 aa, Aspergillus niger
MVSMSALRHGLGVLYLASWLGSSLAASTEQWKSRSIYQTMTDRFARTDGSTTSPCNTTEGLYCGGTWRGMINHLDYIQGMGFDAVMISPIIENVEGRVEYGEAYHGYWPVDLYSLNSHFGTHQDLLDLSDALHARDMYLMMDTVINNMAYITNGSDPATHIDYSTLTPFNSSSYYHPYCKITDWNNFTNAQLCQTGDNIVALPDLYTEHAEVQETLSNWAKEVISTYSIDGLRIDAAKHVNPGFLKNFGDALDIFMTGEVLQQEVSTICDYQNNYIGSLPNYPVYYAMLKAFTLGNTSALATQVQSMKNSCNDVTALSSFSENHDVARFASMTHDMALAKNILTFTLLFDGVPMIYQGQEQHLDGPGSPENREAIWLSEYNTDAELYKLIGKLNAIRKHAYRLDNHYPDVETYPIFEGGSELGFRKGIEGRQVVMLLSTQGTNSSAYNLSMPVSFTGGTVVTEILNCVNYTVNTQSELVVPMDKGEPRVFFPADLMPGSGLCGLPVANVTYAALRTQGAAAAEAALSLGIKTDAASSALLSLGLSVVAGLIVGMW

SEQ ID NO: 4 - DNA, 1890 bp, Aspergillus niger
ttgcttgtcataattcattcggtcacccaagcccacccacgggaagcagaacctccacaagggggcacagagggttgatcgagggtagtactgccaatgatgtttcgaaaatccgcttccctcctgggccaacggctcatggccgtttgtctcctgtgctggtgcgtttcgctagcgaccgccgcaagcacagaagaatggaagacgcgatccatctaccagacgatgacggatcggtttgccctcaccaacggttcgacgaccgcaccatgcaatactacggtagccaactactgtggcgggtcttggcaggggacgatcgataagctggactacatccaaggcatggggttcgatgcgatcatgatctctcccgtaattaaaaacattgcggggcgatctaaggacggtgaggcctaccatggatactggcctctggatctatacgagatcaattctcatttcggaacccgggaggaactgttgaagctgagcgaggagatccacgcacgcggcatgtacttgctgctggacgtcgtcatcaataatatggcttacatgacggacggcgaggatcctgcgacgaccattgactacaatgttttccctcagttcaatggatcttcatacttccacccctactgtcttattacgaactggaataactatacggacgcgcagtggtgtcagactggtgacaattatactgcactcccagatctgtacacagaacacaccgcggtgcagaacatcttaatggactggagtaaatcggtcatcagcaattactccgtcgacgggctacgaatcgacgctgccaagtccctcactcccagctttctgcctacatatgcgagtaccgtgggcggattcatgaccggcgaagtcatggattcgaacgccaccaacgtgtgcaaatatcagacggattacctgccaagtcttccaaactaccccctctattactccatgatcacggccttcctcaacggcgagcctgcgaccttgctcgaggaaatcgcgacgatcaatgatctttgccctgatacgttcgcaatggtcaacttcatcgaagatcaagacgttgaccgatgggcctacatgaatgacgacatcatgctagctaaaactgcactgaccttcatgatgctctacgacggtattcccttggtctaccagggtctggagcaggccattgcctattccaaccgagcggccttgtggttgacagatttcgacaccaatgcgacgctttataaacacatcaagaaactcaatgccatccgcaaacatgccattaaccttgattccagctacatcagttcgaaaacatatcccatctatcaaggaggtagcgagttggctttctggaagggcaacaatggacgccaagtcatcatggttttatctacggcgggctcgaatggttcagcttatactctgacactacccgtaagctatggggctagtgaggtggtcacggaagtgctgaactgcgtcaattatacggtcaacacttacagtcaattggtcgtggacatggataagggcgaacctcgggtcttctttcccgcttcgatgatgccggggagcgggctgtgtggctacaatacttctaatgtgacgtattctgagctgagacttgctgctgtgggatcttcgtcgtctgctggtagccactctgtcataccgtctgcttttgcttcgcttttcatggcgatagttgcatttttggcattccggatataagttcatattttttctactcagttttataccagcaaataaaacgatgtcctatgaatatacacatttcaaaagtggagacaaaattatatctttatatccctagcacctccctttccgtatcctttcctgcaataaagacct

SEQ ID NO: 5 - DNA, 1650 bp, Aspergillus niger
atgtttcgaaaatccgcttccctcctgggccaacggctcatggccgtttgtctcctgtgctggtgcgtttcgctagcgaccgccgcaagcacagaagaatggaagacgcgatccatctaccagacgatgacggatcggtttgccctcaccaacggttcgacgaccgcaccatgcaatactacggtagccaactactgtggcgggtcttggcaggggacgatcgataagctggactacatccaaggcatggggttcgatgcgatcatgatctctcccgtaattaaaaacattgcggggcgatctaaggacggtgaggcctaccatggatactggcctctggatctatacgagatcaattctcatttcggaacccgggaggaactgttgaagctgagcgaggagatccacgcacgcggcatgtacttgctgctggacgtcgtcatcaataatatggcttacatgacggacggcgaggatcctgcgacgaccattgactacaatgttttccctcagttcaatggatcttcatacttccacccctactgtcttattacgaactggaataactatacggacgcgcagtggtgtcagactggtgacaattatactgcactcccagatctgtacacagaacacaccgcggtgcagaacatcttaatggactggagtaaatcggtcatcagcaattactccgtcgacgggctacgaatcgacgctgccaagtccctcactcccagctttctgcctacatatgcgagtaccgtgggcggattcatgaccggcgaagtcatggattcgaacgccaccaacgtgtgcaaatatcagacggattacctgccaagtcttccaaactaccccctctattactccatgatcacggccttcctcaacggcgagcctgcgaccttgctcgaggaaatcgcgacgatcaatgatctttgccctgatacgttcgcaatggtcaacttcatcgaagatcaagacgttgaccgatgggcctacatgaatgacgacatcatgctagctaaaactgcactgaccttcatgatgctctacgacggtattcccttggtctaccagggtctggagcaggccattgcctattccaaccgagcggccttgtggttgacagatttcgacaccaatgcgacgctttataaacacatcaagaaactcaatgccatccgcaaacatgccattaaccttgattccagctacatcagttcgaaaacatatcccatctatcaaggaggtagcgagttggctttctggaagggcaacaatggacgccaagtcatcatggttttatctacggcgggctcgaatggttcagcttatactctgacactacccgtaagctatggggctagtgaggtggtcacggaagtgctgaactgcgtcaattatacggtcaacacttacagtcaattggtcgtggacatggataagggcgaacctcgggtcttctttcccgcttcgatgatgccggggagcgggctgtgtggctacaatacttctaatgtgacgtattctgagctgagacttgctgctgtgggatcttcgtcgtctgctggtagccactctgtcataccgtctgcttttgcttcgcttttcatggcgatagttgcatttttggcattccggatataa

SEQ ID NO: 6 - protein, 549 aa, Aspergillus niger
MFRKSASLLGQRLMAVCLLCWCVSLATAASTEEWKTRSIYQTMTDRFALTNGSTTAPCNTTVANYCGGSWQGTIDKLDYIQGMGFDAIMISPVIKNIAGRSKDGEAYHGYWPLDLYEINSHFGTREELLKLSEEIHARGMYLLLDVVINNMAYMTDGEDPATTIDYNVFPQFNGSSYFHPYCLITNWNNYTDAQWCQTGDNYTALPDLYTEHTAVQNILMDWSKSVISNYSVDGLRIDAAKSLTPSFLPTYASTVGGFMTGEVMDSNATNVCKYQTDYLPSLPNYPLYYSMITAFLNGEPATLLEEIATINDLCPDTFAMVNFIEDQDVDRWAYMNDDIMLAKTALTFMMLYDGIPLVYQGLEQAIAYSNRAALWLTDFDTNATLYKHIKKLNAIRKHAINLDSSYISSKTYPIYQGGSELAFWKGNNGRQVIMVLSTAGSNGSAYTLTLPVSYGASEVVTEVLNCVNYTVNTYSQLVVDMDKGEPRVFFPASMMPGSGLCGYNTSNVTYSELRLAAVGSSSSAGSHSVIPSAFASLFMAIVAFLAFRI

SEQ ID NO: 7 - DNA, 2520 bp, Aspergillus niger
tgctcaacaggaggttacgcaggtcataaccatcaagacattttttctcacctctttccagctaataatcccaccattgaactactctccttgccttctttcatctgcgcatgcaaacgccaccaaccatggacgacgggtggtggatcatctcgttgctggtggtaactctgggaatccccacggttaatgcggcttccagagaccaatggatcggtcgctctatctatcaaatcgtgaccgatcgctttgctcgatcggataactcaaccaccgctgcttgtgatgcagcactaggaaactactgcggaggctctttccagggcatcatcaataagctggactacatccaggagctcgggtttgatgcggtaggtttaccaccatcatcctgcctctcgctctctgccactctttctcatacctgaaacaccagatatggatctctcccgcacaaagccaaatttccgcccgaacagcagatctctcaggtatggcactttcaaaatcttcacaaaccatctgcaactctgtacattgttatatacacgatatactatgtagcttggtaggtaagaaattcaacatgttaacggttcatagcataccatggatattggcccaatgatctgtattccatcaactctcattttggcactcccaaggagctggaagccttgtcctctgccctgcacgatcgtggcatggtgagtcgctctgccattttccctaaagatgacactgaccgcgcaaaacccagtacttgatgcttgacatcgtggttggtgatatggcctgggcgggaaatcacagcaccgtcgattacagcaactttaatcctttcaatgatcagaagtttttccatgatttcaagctcctctccagtgaccccacaaatgaaacttgtgttctggatgtaagtctgtattgtgaatcatgacccgtgggattttgctaaccccatatgaagtgctggatgggagacaccgtcgtatcacttcccgatctgcgaaatgaagaccaacaagttcagaatattcttggtacctggatttcggggttggtttcgaactactcaagtaagacttcttccttggccgttatggaatattgattgacctgtttctgttttgtagttgacggactgcgtattgacagcgtcttaaatatcgccccggacttcttctccaacttcaccaagtcatcaggggttttcactgtcggtgaaggtgccacggccgatgcggctgatgtttgccctctgcagccaagtttaaatgggcttttgaattatccattgtaagtaccgcatcgtggtgagtaaagtcagtcgtgttgactttctcaggtactatattcttaccgacgccttcaacacgaccaacgggaacctgagcaccattaccgagtccataagctacaccaaaggacagtgcgaggtagggcatgccttcgaattggattataagggcaaactaatcatatacaggatgtcttggctctggggacgtttactgcaaaccaggatgttccgcgcttcggttcatacacgtcggatatttcggtaagttcagtcggtggtcctatcttggtactgaacaatgtttgactttattgcttgtgagcagctggcgcgcaatatcctgaccagcagtatgctgacggacggcatccccatccgtatgttccgatccccgcctcccccatagtaagcaagattcctcagacaccactgataatcaaaatacagtctattacggcgaggagcagcatttgacaggatcctacaatcctgtcaaccgtgaggctctgtggctgaccaactactcgatgcgctcgacctccctccccaccctcgtccaatccctgaaccgccttcgatcgtatgctagcggggacggtgagcagtacacgcaaaagtcgcaatctgggagcgattacctctcgtacctgtcagcacccatttacaattctacgcacattctggccacgcgcaaggggtttgcgggcaatcagatcgtgagtgtcgtatccaatctgggagccaagccagccagcaaagccaccacgaaaatcacgttgggctcagacgagactggattccaatccaagcagaatgtgaccgagatcttatcgtgcaagacgtatgtcacggattccagcgggaatctggcagtggatctgagctcggacgggggtccgcgcgtgtactatcccacggacagcctgaaggacagcactgatatctgtggtgatcagaccaaatcggcgaccccgagtagctccgcagcctcgtccgcgagcttgacccagtccaagggttcagagacctgtttgtttggggtgccgttggggataagcacattggtggtcacagttgcgatggccacgtcctacgtgttctagtctccttgagcctctaccccttctgtttgtcgcctgtctttccctgtttcgcattacccctatgattattgtttgctcataacgctcttttgaatgttttgaaactgtggcttt

SEQ ID NO: 8 - DNA, 1704 bp, Aspergillus niger
atggacgacgggtggtggatcatctcgttgctggtggtaactctgggaatccccacggttaatgcggcttccagagaccaatggatcggtcgctctatctatcaaatcgtgaccgatcgctttgctcgatcggataactcaaccaccgctgcttgtgatgcagcactaggaaactactgcggaggctctttccagggcatcatcaataagctggactacatccaggagctcgggtttgatgcgatatggatctctcccgcacaaagccaaatttccgcccgaacagcagatctctcagcataccatggatattggcccaatgatctgtattccatcaactctcattttggcactcccaaggagctggaagccttgtcctctgccctgcacgatcgtggcatgtacttgatgcttgacatcgtggttggtgatatggcctgggcgggaaatcacagcaccgtcgattacagcaactttaatcctttcaatgatcagaagtttttccatgatttcaagctcctctccagtgaccccacaaatgaaacttgtgttctggattgctggatgggagacaccgtcgtatcacttcccgatctgcgaaatgaagaccaacaagttcagaatattcttggtacctggatttcggggttggtttcgaactactcaattgacggactgcgtattgacagcgtcttaaatatcgccccggacttcttctccaacttcaccaagtcatcaggggttttcactgtcggtgaaggtgccacggccgatgcggctgatgtttgccctctgcagccaagtttaaatgggcttttgaattatccattgtactatattcttaccgacgccttcaacacgaccaacgggaacctgagcaccattaccgagtccataagctacaccaaaggacagtgcgaggatgtcttggctctggggacgtttactgcaaaccaggatgttccgcgcttcggttcatacacgtcggatatttcgctggcgcgcaatatcctgaccagcagtatgctgacggacggcatccccatcctctattacggcgaggagcagcatttgacaggatcctacaatcctgtcaaccgtgaggctctgtggctgaccaactactcgatgcgctcgacctccctccccaccctcgtccaatccctgaaccgccttcgatcgtatgctagcggggacggtgagcagtacacgcaaaagtcgcaatctgggagcgattacctctcgtacctgtcagcacccatttacaattctacgcacattctggccacgcgcaaggggtttgcgggcaatcagatcgtgagtgtcgtatccaatctgggagccaagccagccagcaaagccaccacgaaaatcacgttgggctcagacgagactggattccaatccaagcagaatgtgaccgagatcttatcgtgcaagacgtatgtcacggattccagcgggaatctggcagtggatctgagctcggacgggggtccgcgcgtgtactatcccacggacagcctgaaggacagcactgatatctgtggtgatcagaccaaatcggcgaccccgagtagctccgcagcctcgtccgcgagcttgacccagtccaagggttcagagacctgtttgtttggggtgccgttggggataagcacattggtggtcacagttgcgatggccacgtcctacgtgttctag

SEQ ID NO: 9 - protein, 567 aa, Aspergillus niger
MDDGWWIISLLVVTLGIPTVNAASRDQWIGRSIYQIVTDRFARSDNSTTAACDAALGNYCGGSFQGIINKLDYIQELGFDAIWISPAQSQISARTADLSAYHGYWPNDLYSINSHFGTPKELEALSSALHDRGMYLMLDIVVGDMAWAGNHSTVDYSNFNPFNDQKFFHDFKLLSSDPTNETCVLDCWMGDTVVSLPDLRNEDQQVQNILGTWISGLVSNYSIDGLRIDSVLNIAPDFFSNFTKSSGVFTVGEGATADAADVCPLQPSLNGLLNYPLYYILTDAFNTTNGNLSTITESISYTKGQCEDVLALGTFTANQDVPRFGSYTSDISLARNILTSSMLTDGIPILYYGEEQHLTGSYNPVNREALWLTNYSMRSTSLPTLVQSLNRLRSYASGDGEQYTQKSQSGSDYLSYLSAPIYNSTHILATRKGFAGNQIVSVVSNLGAKPASKATTKITLGSDETGFQSKQNVTEILSCKTYVTDSSGNLAVDLSSDGGPRVYYPTDSLKDSTDICGDQTKSATPSSSAASSASLTQSKGSETCLFGVPLGISTLVVTVAMATSYVF

(19) United States
(12) Patent Application Publication
(10) Pub. No.: US 2010/0317065 A1
(43) Pub. Date: Dec. 16, 2010

(54) GLUCANOTRANSFERASE

(75) Inventors: Rachel Maria Van Der Kaaij, Eelde (NL); Marc Jos Elise Cornelis Van Der Maarel, Haren (NL); Lubbert Dijkhuizen, Zuidlaren (NL)

Correspondence Address: NIXON & VANDERHYE, PC, 901 NORTH GLEBE ROAD, 11TH FLOOR, ARLINGTON, VA, 22203, US

(73) Assignee: DSM IP ASSETS B.V., Heerlen (NL)

(21) Appl. No.: 12/445,818

(22) PCT Filed: Oct. 17, 2007
(86) PCT No.: PCT/EP2007/061081

§ 371 Date: Aug. 4, 2009

(30) Foreign Application Priority Data
Oct. 19, 2006 (EP) 06122600.7

(51) Int. Cl. C12P 19/18; C12N 9/10; C07H 21/04; C12N 15/63; C12N 1/15

(52) U.S. Cl. 435/97; 435/193; 536/23.2; 435/320.1; 435/254.3

(57) ABSTRACT

The present invention describes an isolated polypeptide which has glucanotransferase activity, selected from the group consisting of: a) a polypeptide which has an amino acid sequence which has at least 40% amino acid sequence identity with amino acids 1 to 555, 1 to 549 or 1 to 567 of SEQ ID NO: 3 or 6 or a fragment thereof; b) a polypeptide which is encoded by a polynucleotide which hybridizes under low stringency conditions with (i) the nucleic acid sequence of SEQ ID NO: 1, 2, 4 or 5 which is at least 80% or 90% identical over 60, preferably over 100 nucleotides, more preferably at least 90% identical over 200 nucleotides, or (ii) a nucleic acid sequence complementary to the nucleic acid sequence of SEQ ID NO: 1, 2, 4 or 5.

### FIELD OF THE INVENTION

The present invention relates to novel glucanotransferase enzymes.

### BACKGROUND OF THE INVENTION

The α-amylase superfamily comprises a large variation of enzymes that are active towards polysaccharides with α-glucosidic linkages such as starch, glycogen or pullulan. A common property of these enzymes is that they convert their substrate with retention of the α-anomeric configuration. The tertiary structure of these enzymes is characterized by a (β/α)₈ barrel containing four highly conserved amino acids regions that form the catalytic site (MacGregor et al (2001) Biochim Biophys Act 1546, 1-20). Members of the α-amylase superfamily form clan GH-H and are clustered in Glycosyl Hydrolase (GH) families 13,70 and 77 (Henrissat (1991) The Biochem J 280, 309-316; Henrissat & Bairoch (1996) Biochem J Lett 316, 695-696). Most of the members of family GH13 cleave an α-glycosidic linkage using water as an acceptor molecule. The best known hydrolytic enzyme of family GH13 is α-amylase (E.C. 3.2.1.1), which hydrolysis the internal α-1,4-glycosidic bonds in starch, glycogen and maltooligosaccharides. Other GH13 family members are also capable to perform a glucanotransferase type of reaction in addition to the hydrolytic action. In the transferase type reaction the enzyme can use oligosaccharides as acceptors to from new α-glycosidic linkages. Well studied examples of α-glucanotransferases are cyclomaltodextrin glucanotransferase (E.C. 2.4.1.19; van der Veen et al (2000) Biochim Biophys Act 1543, 336-360) and amylomaltase (E.C. 2.4.1.25; Meissner & Liebl (1998) Eur J Biochem 258, 1050-1058). The bacterial amylomaltases are divided over family GH13 and GH70, based on the conservation of amino acids in the active site.

It is well known that the fungus Aspergillus niger produces a number of extracellular starch degrading enzymes, such as acid amylase and glucoamylase. In addition, two other alpha-amylase enzymes (AmyA/B; Korman (1990) Curr. Gen 17, 203-212) and an α-glucosidase (Nagamine et al (2003) Biosci Biotechnol Biochem 67, 2194-2202) have previously been identified in Aspergillus niger. In the recently published A niger genome and the A niger genome available via DSM, The Netherlands, additional GH13 family members can be revealed, as judged by sequence similarity and the presence of the four conserved α-amylase regions. Three of these newly identified enzymes showed a typical glycosylphosphatidylinositol (GPI) anchoring sequence at the C-terminus, indicating attachment of the protein to the cell wall or cell membrane. Recently a homologue of these GPI-anchored A niger enzymes was described in Schizosaccheromyces pombe (Morita et al (2006) Biosci Biotechnol Biochem 70, 1454-1463). The authors showed that these enzymes were indeed anchored to the membrane and cell wall, but were unable to demonstrate its catalytic activity. In Aspergillus fumigatus, two GPI-anchored β-glucanosyltransferases are described (Mouyna et al (2000) J Biol Chem 19; 275, 14882-14889) which play a role in the Aspergillus cell wall maintenance by altering the structure of the 8-1,3-glucanpolymer. These enzymes that are able to modify the α-1,3-glucan structure, which structure is also present in the Aspergillus cell wall.

Starch modification is of high important to e.g. the food industry. As used herein, the term ‘starch’ comprises both native starch and non-substituted starch derivatives. By the latter are meant starches obtained by partially breaking down native starch through acid and/or enzymatic hydrolysis to obtain a DE of not more that 5, because otherwise the polymeric character of starch is lost. Starch is composed of a mixture of amylose and amylopectin; the ratio between the two varies between starch sources. Examples of starches are potato starch, maize starch, wheat starch, rice starch and tapioca starch and derivatives thereof. It is known that starch can be modified in many different ways. Starch products are produced for various uses (EP0932444), and the starch modification that is carried out depends on the specific application. Many of the known starch modifications lead to the production of relatively viscous solutions. When high viscosity is not obtained, the molecular weight of the starch has usually decreased significantly, leading to loss of typical starch characteristics. Enzymatic starch modification is commonly used in the industry for a variety purposes, including modification of its texturizing properties. Examples include the use of α-1,6-D-glucanohydrolase as described in U.S. Pat. No. 4,971,723 and the use of α-1,4-glucosyl transferase (E.C. 2.4.1.25) as described in EP0932444. The latter patent describes the preparation of a thermo reversible gel from an amylose-containing starch by enzymatic treatment, in which several starch characteristics like the average molecular weight, the reducing power (DE) and the branching percentage are hardly affected. The thermo-reversible gels that can be generated with such starches are of interest to a large variety of application area's, such as in food stuffs, cosmetics, pharmaceutics, detergents, adhesives and drilling fluids. Clearly, enzymes with sugar transferase activity are of interest to the starch and food industry.

Transferase enzymes are also of interest for the production of oligomeric sugar molecules. In such process, a sugar molecule is transferred from a donor to an acceptor molecule, in the end leading to the formation of a new oligomeric sugar molecule. Such oligomeric sugars are of interest to the food industry because of their potential pre-biotic activity. A well known example is the generation of galactose oligomers from the di-sugar lactose using the galactose transferase activity of the enzyme β-galactosidase, but other examples are also described (see e.g. Prapulla et al (2000) Adv Appl Microbiol 47, 299-343). The galactose oligomers (GOS) are generally recognized as effective prebiotic substances as well as other oligosaccharides such as glucoseoligosaccharides. Sugar transferases that can produce oligomeric sugars are therefore useful for the food industry.

Aqueous systems are preferred by the food industry over systems containing organic solvents. In aqueous systems, sugar transferases face a mixture of acceptor molecules competing for the sugar that is to be transferred. In order to form oligosaccharides, the acceptor molecule must be a monomeric or an oligomeric sugar molecule. Most enzymes, however, can also use water as the acceptor molecule. When this is the case, the enzyme performs a simply hydrolysis reaction resulting in the formation of free monomeric sugars and the expense of the formation of oligomeric sugars. This is undesired, and therefore enzymes that do prefer monomeric or oligomeric sugar molecules instead of water as the acceptor molecule are of particular interest.

### DESCRIPTION OF THE INVENTION

We have surprisingly found that A. niger has 4-α-glucanotransferase activity and noted that 3 enzymes code for AgtA, AgtB and AgtC, that belong to the fungal α-amylase family, are in fact 4-α-glucanotransferase enzymes. The enzymes are unique in that they show almost exclusively glucose transferase activity and almost no release of free glucose. The enzymes are able to transfer glucose moieties from starch to a variety of sugar acceptors.

The present invention provides an isolated polypeptide which has 4-α-glucanotransferase activity, selected from the group consisting of:
(a) a polypeptide which has an amino acid sequence which has at least 60% amino acid sequence identity with amino acids 1 to 555, 1 to 549 or 1 to 567 of SEQ ID NO: 3, 6 or 9, respectively, and
(b) a polypeptide which is encoded by a polynucleotide which hybridizes under low stringency conditions with (i) the nucleic acid sequence of SEQ ID NO: 1, 2, 4, 5, 7 or 8 which is at least 80% or 90% identical over 60, preferably over 100 nucleotides, more preferably at least 90% identical over 200 nucleotides, or (ii) a nucleic acid sequence complementary to the nucleic acid sequence of SEQ ID NO: 1, 2, 4, 5, 7 or 8.

Preferably the polypeptide which is obtained from a fungus, more preferably an Aspergillus, most preferably from Aspergillus niger. The present invention also relates to a nucleic acid construct comprising the polynucleotide operably linked to one or more control sequences that direct the production of the polypeptide in a suitable expression host, as well as the recombinant host cell comprising the nucleic acid construct. According to another aspect of the invention a polypeptide is provided which has 4-α-glucanotransferase activity and which is able to use as donor substrates amylose containing starch and glucose-polymers containing α-(1,4) glycosidic bonds and consisting of at least five anhydroglucose units and as acceptor molecules maltose, maltooligosaccharides, nigeran, nigerotriose or β-(1,3)-glucan.

Moreover the present invention provides a polypeptide having 4-α-glucanotransferase activity and which is able to use as donor substrates amylose containing starch or glucose-polymers containing α-(1,4) glycosidic bonds and consisting of at least five anhydroglucose units and as acceptor molecules maltose, maltooligosaccharides, nigeran, nigerotriose or β-(1,3)-glucan and in which the transfer-percentage of sugars in aqueous solution to acceptor molecules other than water is higher than 90%, preferably higher than 95%. By transfer-percentage is meant the percentage of the amount of glucose moiety transferred to maltose, maltooligosaccharide, nigeran, nigerotriose or β-(1,3)-glucan relative to the amount of glucose moiety freed from amylose containing starch or glucose-polymers containing α-(1,4) glycosidic bonds and consisting of at least five anhydroglucose units.

According to another aspect of the invention a method is disclosed for the transfer of a glucose moiety in a medium which medium comprises

(a) amylose containing starch or glucose-polymers containing α-(1,4) glycosidic bonds and consisting of at least five anhydroglucose units; and

(b) maltose, maltooligosaccharide, nigeran, nigerotriose or β-(1,3)-glucan,

whereby a polypeptide having 4-α-glucanotransferase activity is used to transfer the glucose moiety from amylose containing starch or glucose-polymers containing α-(1,4) glycosidic bonds and consisting of at least five anhydroglucose units to an acceptor molecule maltose, maltooligosaccharide, nigeran, nigerotriose or β-(1,3)-glucan using. Furthermore the present invention provides the use of a polypeptide of the invention in the preparation of food or feed, a process for treating starch which comprises bringing the starch together with a polypeptide of the invention for a period of time to treat the starch, a process for producing a food or feed which comprises bringing starch together with a polypeptide of the invention for a period of time to treat the starch and using this starch to produce the food or feed, a process for producing an oligosaccharide which comprises bringing starch together for a period of time with a polypeptide of the invention in the presence of a suitable acceptor substrate selected from maltose, maltose-oligosaccharides, nigeran, nigerotriose and α-(1,3)-glucan to form oligosaccharides and a process for producing a food or feed which comprises bringing starch together for a period of time with a polypeptide of the invention in the presence of a suitable acceptor substrate selected from maltose, maltose-oligosaccharides, nigeran, nigerotriose and α-(1,3)-glucan to form oligosaccharides and using these oligosaccharides to produce the food or feed.

### DETAILED DESCRIPTION OF THE INVENTION

A. niger is a well-known producer of starch-degrading enzymes such as acid amylase and glucoamylase. Although these enzymes and their production by A. niger have been studied in much detail, little is known about the complete set of enzymes of A. niger that can act on starch or the closely related intracellular storage compound glycogen. Most of the enzymes we know to date that act on starch are grouped in the family GH13, of which α-amylase is the best known representative. We screened the complete genome sequence of the A. niger using a family GH13 specific HMMR profile. This revealed three sequences which had a C-terminal GPI-anchoring sequence which was not found in the other family members. All the GPI-anchored family GH13 enzymes which were identified in the fungal genomes were annotated as alpha-amylases, because of their high similarity with known extracellular fungal amylases. However, we performed a detailed analysis of the three A. niger gene sequences which surprisingly revealed the absence of two amino acids commonly preserved in family GH13 enzymes (table 1, see also example 1). The residue His143 in conserved region I is absent in all three sequences as well as in the homologous GPI-anchored proteins found in other fungi.

This amino acid is supposed to be one of the eight invariant residues in the family GH13 (Jespersen et al (1991) Biochem J 280, 51-55). The second generally conserved His residue, His317 in conserved region IV, is present in only one of the three A. niger enzymes (AgtA, AgtB, AgtC), while it is replaced by Gln in the other two. Although His 317 is overall highly conserved in the alpha-amylase family, this residue seems to be less important for the determination of the catalytic activity because several alpha-amylases, e.g. that of Lipomyces starkeyi (Kang et al (2004) FEMS Microbiol Lett 233, 53-64) and Streptomyces hygroscopicus (Hoshiko et al (1987) J Bacteriol 169, 1029-1036) also do not posses a H is in this position.

AgtA and AgtB, two of the identified family GH13 members, were overproduced in A. niger and subsequently purified for a more detailed characterization. We show that both AgtA and AgtB perform a glucanotransferase reaction (see examples for experimental details). Incubations with different substrates showed that the donor molecule for both enzymes needs a minimum of 5 glucose residues, while the acceptor molecule can be as small as maltose. The formation of large oligosaccharides up to DP 30 from maltopentaose by both enzymes indicated that they used reaction products in subsequent reaction rounds, and that they can accept large molecules as donor and/or acceptor molecules (DP=degree of polymerisation). Although the two enzymes perform essentially the same reaction, HPLC analysis of the reaction products showed that AgtA only produces maltooligosaccharides with α-(1,4) glycosidic bonds, while AgtB also produces oligosaccharides with α-(1,6) glycosidic bonds such as isomaltose, isomaltotriose and panose. Using the combination of starch as a donor and a small acceptor molecule it was found that also nigerose and nigerotriose, with α-(1,3)-glycosidic linkages, were used as an acceptor by AgtA and, less efficiently, by AgtB. The enzymatic activity of AgtA and AgtB is unique among the α-glucano-transferases from bacteria as well as eukarya. The α-glucanotransferases that have been described until now usually release one glucose molecule for every transfer event (Kaper et al (2004) Biochem Soc Trans 32, 279-282; Takaha et al (1993) J Biol Chem 268, 1391-1396). Although a small amount of glucose (less than 5% total sugars) was formed by AgtB and to a lesser extent by AgtA (see examples for details), there was no indication that this happened in a 1:1 ratio with each transfer event, as is usually observed for transferase enzymes. AgtA and AgtB also differ from known enzymes with respect to their substrate specificity. Bacterial amylomaltases and the plant D-enzymes generally use molecules as small as maltotriose as a donor and glucose as an acceptor substrate. In contrast, A. niger AgtA and AgtB prefer oligosaccharides with a minimum length of 5 glucose residues as a donor while they can use maltose as acceptor substrate. Additionally, the use of the α-(1,3) linked oligosaccharides nigerose and nigerotriose as acceptor substrate by α-glucanotransferases was never reported before. The A. niger α-glucanotransferases represent a new subgroup among the α-glucanotransferases because of their typical donor and acceptor profile and their C-terminal GPI-anchoring sequence. Based on their common location and amino acid sequences, it is expected that the closely related GPI-anchored family GH13 proteins in other fungi will show similar glucanotransferase activities, although their precise substrate and products profiles, might be slightly different.

AgtA and its homologues are most probably involved in cell wall synthesis in Aspergillus niger. The cell wall of Aspergilli consists of a dense layer of chitin and glucans, covered with mannoproteins at the outer layer (Barnard et al (2001) Med Mycol 39, suppl 1:9-17). The main component of the glucan layer is alkali insoluble β-glucan, with mainly 6-(1,3)-glycosidic bonds (Fontaine et al (2000) J Biol Chem 275, 41528). In addition, an alkali soluble fraction of α-(1,3)-glucan is present, with smaller amounts of nigeran (a glucan with alternating α-(1,3) and α-(1,4) glycosidic bonds). By using starch as the donor substrate we could show that nigerose and nigerotriose can act as acceptor substrates for AgtA and to a lesser extent for AgtB (see examples). This indicated that AgtA could perform a transferase reaction involving an α-(1,4) linked donor substrate and an α-(1,3) linked acceptor, a reaction which was described to occur in S. pombe cell walls (Grun et al (2005) Glycobiol 15, 245-257).

AgtA and AgtB have homologues in other fungi containing cell wall α-glucan. They act on α-(1,4) linked oligo- and polysaccharides, although the existence of this type of polymers in filamentous fungi is not widely described. The discovery of GPI-anchored α-glucanotransferases in a wide range of fungi provides new possibilities to study this part of fungal physiology. Ultimately, as for cell wall β-glucan, this knowledge may lead to new applications in pharmacy and biotechnology by using the enzymes as targets for anti-fungal agents.

A polypeptide of the invention which has glucotransferase may be in an isolated form. As defined herein, an isolated polypeptide is an endogenously produced or a recombinant polypeptide which is essentially free from other non-glucotransferase polypeptides, and is typically at least about 20% pure, preferably at least about 40% pure, more preferably at least about 60% pure, even more preferably at least about 80% pure, still more preferably about 90% pure, and most preferably about 95% pure, as determined by SDS-PAGE. The polypeptide may be isolated by centrifugation and chromatographic methods, or any other technique known in the art for obtaining pure proteins from crude solutions. It will be understood that the polypeptide may be mixed with carriers or diluents which do not interfere with the intended purpose of the polypeptide, and thus the polypeptide in this form will still be regarded as isolated. It will generally comprise the polypeptide in a preparation in which more than 20%, for example more than 30%, 40%, 50%, 80%, 90%, 95% or 99% by weight of the proteins in the preparation is a polypeptide of the invention.

Preferably, the polypeptide of the invention is obtainable from a microorganism which possesses a gene encoding an enzyme with glucotransferase activity. More preferably the microorganism is fungal, and optimally is a filamentous fungus. Preferred organisms are thus of the genus Aspergillus, such as those of the species Aspergillus niger.

In a first embodiment, the present invention provides an isolated polypeptide having an amino acid sequence which has a degree of amino acid sequence identity to amino acids 1 to 555, 1 to 549 or 1 to 567 of SEQ ID NO: 3, 6 or 9 (i.e. the polypeptide), respectively, of at least about 40%, preferably at least about 50%, preferably at least about 60%, preferably at least about 65%, preferably at least about 70%, more preferably at least about 80%, even more preferably at least about 90%, still more preferably at least about 95%, and most preferably at least about 97%, and which has glucotransferase activity.

For the purposes of the present invention, the degree of identity between two or more amino acid sequences is determined by BLAST P protein database search program (Altschul et al., 1997, Nucleic Acids Research 25: 3389-3402) with matrix Blosum 62 and an expected threshold of 10.

A polypeptide of the invention may comprise the amino acid sequence set forth in SEQ ID NO: 3, 6 or 9 or a substantially homologous sequence, or a fragment of either sequence having glucotransferase activity. In general, the naturally occurring amino acid sequence shown in SEQ ID NO: 3, 6 or 9 is preferred.

The polypeptide of the invention may also comprise a naturally occurring variant or species homologue of the polypeptide of SEQ ID NO: 3, 6 or 9.

A variant is a polypeptide that occurs naturally in, for example, fungal, bacterial, yeast or plant cells, the variant having glucotransferase activity and a sequence substantially similar to the protein of SEQ ID NO: 3, 6 or 9. The term “variants” refers to polypeptides which have the same essential character or basic biological functionality as the glucotransferase of SEQ ID NO: 3, 6 or 9, and includes allelic variants. The essential character of glucotransferase of SEQ ID NO: 3, 6 or 9 is that it is an enzyme capable of cleaving the amino-terminal amino acid from a protein or (poly)peptide. Preferably, a variant polypeptide has at least the same level of glucotransferase activity as the polypeptide of SEQ ID NO: 3, 6 or 9. Variants include allelic variants either from the same strain as the polypeptide of SEQ ID NO: 3, 6 or 9 or from a different strain of the same genus or species.

Similarly, a species homologue of the inventive protein is an equivalent protein of similar sequence which is an glucotransferase and occurs naturally in another species of Aspergillus.

Variants and species homologues can be isolated using the procedures described herein which were used to isolate the polypeptide of SEQ ID NO: 3, 6 or 9 and performing such procedures on a suitable cell source, for example a bacterial, yeast, fungal or plant cell. Also possible is to use a probe of the invention to probe libraries made from yeast, bacterial, fungal or plant cells in order to obtain clones expressing variants or species homologues of the polypepetide of SEQ ID NO: 3, 6 or 9. These clones can be manipulated by conventional techniques to generate a polypeptide of the invention which thereafter may be produced by recombinant or synthetic techniques known per se.

The sequence of the polypeptide of SEQ ID NO: 3, 6 or 9 and of variants and species homologues can also be modified to provide polypeptides of the invention. Amino acid substitutions may be made, for example from 1, 2 or 3 to 10, 20 or 30 substitutions. The same number of deletions and insertions may also be made. These changes may be made outside regions critical to the function of the polypeptide, as such a modified polypeptide will retain its glucotransferase activity.

Polypeptides of the invention include fragments of the above mentioned full length polypeptides and of variants thereof, including fragments of the sequence set out in SEQ ID NO: 3, 6 or 9. Such fragments will typically retain activity as glucotransferase. Fragments may be at least 50, 100 or 200 amino acids long or may be this number of amino acids short of the full length sequence shown in SEQ ID NO: 3, 6 or 9.

Polypeptides of the invention can, if necessary, be produced by synthetic means although usually they will be made recombinantly as described below. Synthetic polypeptides may be modified, for example, by the addition of histidine residues or a T7 tag to assist their identification or purification, or by the addition of a signal sequence to promote their secretion from a cell.

Thus, the variants sequences may comprise those derived from strains of Aspergillus other than the strain from which the polypeptide of SEQ ID NO: 3, 6 or 9 was isolated. Variants can be identified from other Aspergillus strains by looking for glucotransferase activity and cloning and sequencing as described herein. Variants may include the deletion, modification or addition of single amino acids or groups of amino acids within the protein sequence, as long as the peptide maintains the basic biological functionality of the glucotransferase of SEQ ID NO: 3, 6 or 9.

Amino acid substitutions may be made, for example from 1, 2 or from 3 to 10, or 30 substitutions. The modified polypeptide will generally retain activity as glucotransferase. Conservative substitutions may be made; such substitutions are well known in the art. Preferably substitutions do not affect the folding or activity of the polypeptide.

Shorter polypeptide sequences are within the scope of the invention. For example, a peptide of at least 50 amino acids or up to 60, 70, 80, 100, 150 or 200 amino acids in length is considered to fall within the scope of the invention as long as it demonstrates the basic biological functionality of the glucotransferase of SEQ ID NO: 2. In particular, but not exclusively, this aspect of the invention encompasses the situation in which the protein is a fragment of the complete protein sequence.

In a second embodiment, the present invention provides an to isolated polypeptide which has glucotransferase activity, and is encoded by polynucleotides which hybridize or are capable of hybrizing under low stringency conditions, more preferably medium stringency conditions, and most preferably high stringency conditions, with (I) the nucleic acid sequence of SEQ ID NO: 1, 2, 4, 5, 7 or 8 or a nucleic acid fragment comprising at least the c-terminal portion of SEQ ID NO: 1, 2, 4, 5, 7 or 8, but having less than all or having bases differing from the bases of SEQ ID NO: 1, 2, 4, 5, 7 or 8; or (ii) with a nucleic acid strand complementary to SEQ ID NO: 1, 2, 4, 5, 7 or 8.

The term “capable of hybridizing” means that the target polynucleotide of the invention can hybridize to the nucleic acid used as a probe (for example, the nucleotide sequence set forth in SEQ. ID NO: 1, 2, 4, 5, 7 or 8, or a fragment thereof, or the complement of SEQ ID NO: 1, 2, 4, 5, 7 or 8) at a level significantly above background. The invention also includes the polynucleotides that encode the glucotransferase of the invention, as well as nucleotide sequences which are complementary thereto. The nucleotide sequence may be RNA or DNA, including genomic DNA, synthetic DNA or cDNA. Preferably, the nucleotide sequence is DNA and most preferably, a genomic DNA sequence. Typically, a polynucleotide of the invention comprises a contiguous sequence of nucleotides which is capable of hybridizing under selective conditions to the coding sequence or the complement of the coding sequence of SEQ ID NO: 1, 2, 4, 5, 7 or 8. Such nucleotides can be synthesized according to methods well known in the art.

A polynucleotide of the invention can hybridize to the coding sequence or the complement of the coding sequence of SEQ ID NO: 1, 2, 4, 5, 7 or 8 at a level significantly above background. Background hybridization may occur, for example, because of other cDNAs present in a cDNA library. The signal level generated by the interaction between a polynucleotide of the invention and the coding sequence or complement of the coding sequence of SEQ ID NO: 1, 2, 4, 5, 7 or 8 is typically at least 10 fold, preferably at least 20 fold, more preferably at least 50 fold, and even more preferably at least 100 fold, as intense as interactions between other polynucleotides and the coding sequence of SEQ ID NO: 1, 2, 4, 5, 7 or 8. The intensity of interaction may be measured, for example, by radiolabelling the probe, for example with ³²P. Selective hybridization may typically be achieved using conditions of low stringency (0.3M sodium chloride and 0.03M sodium citrate at about 40° C.), medium stringency (for example, 0.3M sodium chloride and 0.03M sodium citrate at about 50° C.) or high stringency (for example, 0.3M sodium chloride and 0.03M sodium citrate at about 60° C.).

### Modifications

Polynucleotides of the invention may comprise DNA or RNA. They may be single or double stranded. They may also be polynucleotides which include within them synthetic or modified nucleotides including peptide nucleic acids. A number of different types of modifications to polynucleotides are known in the art. These include a methylphosphonate and phosphorothioate backbones, and addition of acridine or polylysine chains at the 3′ and/or 5′ ends of the molecule. For the purposes of the present invention, it is to be understood that the polynucleotides described herein may be modified by any method available in the art.

It is to be understood that skilled persons may, using routine techniques, make nucleotide substitutions that do not affect the polypeptide sequence encoded by the polynucleotides of the invention to reflect the codon usage of any particular host organism in which the polypeptides of the invention are to be expressed.

The coding sequence of SEQ ID NO: 1, 2, 4, 5, 7 or 8 may be modified by nucleotide substitutions, for example from 1, 2 or 3 to 10, 25, 50 or 100 substitutions. The polynucleotide of SEQ ID NO: 1, 2, 4, 5, 7 or 8 may alternatively or additionally be modified by one or more insertions and/or deletions and/or by an extension at either or both ends. The modified polynucleotide generally encodes a polypeptide which has glucotransferase activity. Degenerate substitutions may be made and/or substitutions may be made which would result in a conservative amino acid substitution when the modified sequence is translated, for example as discussed with reference to polypeptides later.

### Homologues

A nucleotide sequence which is capable of selectively hybridizing to the complement of the DNA coding sequence of SEQ ID NO: 1, 2, 4, 5, 7 or 8 is included in the invention and will generally have at least 50% or 60%, at least 70%, at least 80%, at least 90%, at least 95%, at least 98% or at least 99% sequence identity to the coding sequence of SEQ ID NO: 1, 2, 4, 5, 7 or 8 over a region of at least 60, preferably at least 100, more preferably at least 200 contiguous nucleotides or most preferably over the full length of SEQ ID NO: 1, 2, 4, 5, 7 or 8. Likewise, a nucleotide which encodes an active glucotransferase and which is capable of selectively hybridizing to a fragment of a complement of the DNA coding sequence of SEQ ID NO: 1, 2, 4, 5, 7 or 8, is also embraced by the invention. A C-terminal fragment of the nucleic acid sequence of SEQ ID NO: 1, 2, 4, 5, 7 or 8 which is at least 80% or 90% identical over 60, preferably over 100 nucleotides, more preferably at least 90% identical over 200 nucleotides is encompassed by the invention.

Any combination of the above mentioned degrees of identity and minimum sizes may be used to define polynucleotides of the invention, with the more stringent combinations (i.e. higher identity over longer lengths) being preferred. Thus, for example, a polynucleotide which is at least 80% or 90% identical over 60, preferably over 100 nucleotides, forms one aspect of the invention, as does a polynucleotide which is at least 90% identical over 200 nucleotides.

The UWGCG Package provides the BESTFIT program which may be used to calculate identity (for example used on its default settings).

The PILEUP and BLAST N algorithms can also be used to calculate sequence identity or to line up sequences (such as identifying equivalent or corresponding sequences, for example on their default settings).

Software for performing BLAST analyses is publicly available through the National Center for Biotechnology Information (http://www.ncbi.nlm.nih.gov/). This algorithm involves first identifying high scoring sequence pair (HSPs) by identifying short words of length W in the query sequence that either match or satisfy some positive-valued threshold score T when aligned with a word of the same length in a database sequence. T is referred to as the neighborhood word score threshold. These initial neighborhood word hits act as seeds for initiating searches to find HSPs containing them. The word hits are extended in both directions along each sequence for as far as the cumulative alignment score can be increased. Extensions for the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLAST program uses as defaults a word length (W) of 11, the BLOSUM62 scoring matrix alignments (B) of 50, expectation (E) of 10, M=5, N=4, and a comparison of both strands.

The BLAST algorithm performs a statistical analysis of the similarity between two sequences. One measure of similarity provided by the BLAST algorithm is the smallest sum probability (P(N)), which provides an indication of the probability by which a match between two nucleotide or amino acid sequences would occur by chance. For example, a sequence is considered similar to another sequence if the smallest sum probability in comparison of the first sequence to the second sequence is less than about 1, preferably less than about 0.1, more preferably less than about 0.01, and most preferably less than about 0.001.

### Primers and Probes

Polynucleotides of the invention include and may be used as primers, for example as polymerase chain reaction (PCR) primers, as primers for alternative amplification reactions, or as probes for example labelled with a revealing label by conventional means using radioactive or non-radioactive labels, or the polynucleotides may be cloned into vectors. Such primers, probes and other fragments will be at least 15, for example at least 20, 25, 30 or 40 nucleotides in length. They will typically be up to 40, 50, 60, 70, 100, 150, 200 or 300 nucleotides in length, or even up to a few nucleotides (such as 5 or 10 nucleotides) short of the coding sequence of SEQ ID NO: 1, 2, 4, 5, 7 or 8.

In general, primers will be produced by synthetic means, involving a step-wise manufacture of the desired nucleic acid sequence one nucleotide at a time. Techniques for accomplishing this using automated protocols are readily available in the art. Longer polynucleotides will generally be produced using recombinant means, for example using PCR cloning techniques. This will involve making a pair of primers (typically of about 15-30 nucleotides) to amplify the desired region of the glucotransferase to be cloned, bringing the primers into contact with mRNA, cDNA or genomic DNA obtained from a yeast, bacterial, plant, prokaryotic or fungal cell, preferably of an Aspergillus strain, performing a polymerase chain reaction under conditions suitable for the amplification of the desired region, isolating the amplified fragment (e.g. by purifying the reaction mixture on an agarose gel) and recovering the amplified DNA. The primers may be designed to contain suitable restriction enzyme recognition sites so that the amplified DNA can be cloned into a suitable cloning vector.

Such techniques may be used to obtain all or part of the polynucleotides encoding the glucotransferase sequences described herein. Introns, promoter and trailer regions are within the scope of the invention and may also be obtained in an analogous manner (e.g. by recombinant means, PCR or cloning techniques), starting with genomic DNA from a fungal, yeast, bacterial plant or prokaryotic cell.

The polynucleotides or primers may carry a revealing label. Suitable labels include radioisotopes such as ³²P or ³⁵S, enzyme labels, or other protein labels such as biotin. Such labels may be added to polynucleotides or primers of the invention and may be detected using techniques known to persons skilled in the art.

Polynucleotides or primers (or fragments thereof) labelled or unlabelled may be used in nucleic acid-based tests for detecting or sequencing an glucotransferase or a variant thereof in a fungal sample. Such detection tests will generally comprise bringing a fungal sample suspected of containing the DNA of interest into contact with a probe comprising a polynucleotide or primer of the invention under hybridizing conditions, and detecting any duplex formed between the probe and nucleic acid in the sample. Detection may be achieved using techniques such as PCR or by immobilizing the probe on a solid support, removing any nucleic acid in the sample which is not hybridized to the probe, and then detecting any nucleic acid which is hybridized to the probe. Alternatively, the sample nucleic acid may be immobilized on a solid support, the probe hybridized and the amount of probe bound to such a support after the removal of any unbound probe detected.

The probes of the invention may conveniently be packaged in the form of a test kit in a suitable container. In such kits the probe may be bound to a solid support where the assay format for which the kit is designed requires such binding. The kit may also contain suitable reagents for treating the sample to be probed, hybridizing the probe to nucleic acid in the sample, control reagents, instructions, and the like. The probes and polynucleotides of the invention may also be used in microassay.

Preferably, the polynucleotide of the invention is obtainable from the same organism as the polypeptide, such as a fungus, in particular a fungus of the genus Aspergillus.

The polynucleotides of the invention also include variants of the sequence of SEQ ID NO: 1, 2, 4, 5, 7 or 8 which encode for a polypeptide having glucotransferase activity. Variants may be formed by additions, substitutions and/or deletions. Such variants of the coding sequence of SEQ ID NO: 1, 2, 4, 5, 7 or 8 may thus encode polypeptides which have the glucotransferase activity.

### Production of Polynucleotides

Polynucleotides which do not have 100% identity with SEQ ID NO: 1, 2, 4, 5, 7 or 8 but fall within the scope of the invention can be obtained in a number of ways. Thus, variants of the glucotransferase sequence described herein may be obtained for example, by probing genomic DNA libraries made from a range of organisms, such as those discussed as sources of the polypeptides of the invention. In addition, other fungal, plant or prokaryotic homologues of glucotransferase may be obtained and such homologues and fragments thereof in general will be capable of hybridising to SEQ ID NO: 1, 2, 4, 5, 7 or 8. Such sequences may be obtained by probing cDNA libraries or genomic DNA libraries from other species, and probing such libraries with probes comprising all or part of SEQ ID NO: 1, 2, 4, 5, 7 or 8 under conditions of low, medium to high stringency (as described earlier). Nucleic acid probes comprising all or part of SEQ ID NO: 1, 2, 4, 5, 7 or 8 may be used to probe cDNA or genomic libraries from other species, such as those described as sources for the polypeptides of the invention.

Species homologues may also be obtained using degenerate PCR, which uses primers designed to target sequences within the variants and homologues which encode conserved amino acid sequences. The primers can contain one or more degenerate positions and will be used at stringency conditions lower than those used for cloning sequences with single sequence primers against known sequences.

Alternatively, such polynucleotides may be obtained by site directed mutagenesis of the glucotransferase sequences or variants thereof. This may be useful where, for example, silent codon changes to sequences are required to optimize codon preferences for a particular host cell in which the polynucleotide sequences are being expressed. Other sequence changes may be made in order to introduce restriction enzyme recognition sites, or to alter the property or function of the polypeptides encoded by the polynucleotides.

The invention includes double stranded polynucleotides comprising a polynucleotide of the invention and its complement.

The present invention also provides polynucleotides encoding the polypeptides of the invention described above. Since such polynucleotides will be useful as sequences for recombinant production of polypeptides of the invention, it is not necessary for them to be capable of hybridising to the sequence of SEQ ID NO: 1, 2, 4, 5, 7 or 8, although this will generally be desirable. Otherwise, such polynucleotides may be labelled, used, and made as described above if desired.

### Recombinant Polynucleotides

The invention also provides vectors comprising a polynucleotide of the invention, including cloning and expression vectors, and in another aspect methods of growing, transforming or transfecting such vectors into a suitable host cell, for example under conditions in which expression of a polypeptide of, or encoded by a sequence of, the invention occurs. Provided also are host cells comprising a polynucleotide or vector of the invention wherein the polynucleotide is heterologous to the genome of the host cell. The term “heterologous”, usually with respect to the host cell, means that the polynucleotide does not naturally occur in the genome of the host cell or that the polypeptide is not naturally produced by that cell. Preferably, the host cell is a yeast cell, for example a yeast cell of the genus Kluyveromyces or Saccharomyces or a filamentous fungal cell, for example of the genus Aspergillus.

Polynucleotides of the invention can be incorporated into a recombinant replicable vector, for example a cloning or expression vector. The vector may be used to replicate the nucleic acid in a compatible host cell. Thus, in a further embodiment, the invention provides a method of making polynucleotides of the invention by introducing a polynucleotide of the invention into a replicable vector, introducing the vector into a compatible host cell, and growing the host cell under conditions which bring about replication of the vector. The vector may be recovered from the host cell. Suitable host cells are described below in connection with expression vectors.

### Vectors

The vector into which the expression cassette of the invention is inserted may be any vector that may conveniently be subjected to recombinant DNA procedures, and the choice of the vector will often depend on the host cell into which it is to be introduced. Thus, the vector may be an autonomously replicating vector, i.e. a vector which exists as an extra-chromosomal entity, the replication of which is independent of chromosomal replication, such as a plasmid. Alternatively, the vector may be one which, when introduced into a host cell, is integrated into the host cell genome and replicates together with the chromosome(s) into which it has been integrated.

Preferably, when a polynucleotide of the invention is in a vector it is operably linked to a regulatory sequence which is capable of providing for the expression of the coding sequence by the host cell, i.e. the vector is an expression vector. The term “operably linked” refers to a juxtaposition wherein the components described are in a relationship permitting them to function in their intended manner. A regulatory sequence such as a promoter, enhancer or other expression regulation signal “operably linked” to a coding sequence is positioned in such a way that expression of the coding sequence is achieved under conditions compatible with the control sequences.

The vectors may, for example in the case of plasmid, cosmid, virus or phage vectors, be provided with an origin of replication, optionally a promoter for the expression of the polynucleotide and optionally an enhancer and/or a regulator of the promoter. A terminator sequence may be present, as may be a polyadenylation sequence. The vectors may contain one or more selectable marker genes, for example an ampicillin resistance gene in the case of a bacterial plasmid or a neomycin resistance gene for a mammalian vector. Vectors may be used in vitro, for example for the production of RNA or can be used to transfect or transform a host cell.

The DNA sequence encoding the polypeptide is preferably introduced into a suitable host as part of an expression construct in which the DNA sequence is operably linked to expression signals which are capable of directing expression of the DNA sequence in the host cells. For transformation of the suitable host with the expression construct transformation procedures are available which are well known to the skilled person. The expression construct can be used for transformation of the host as part of a vector carrying a selectable marker, or the expression construct is co-transformed as a separate molecule together with the vector carrying a selectable marker. The vectors may contain one or more selectable marker genes.

Preferred selectable markers include but are not limited to those that complement a defect in the host cell or confer resistance to a drug. They include for example versatile marker genes that can be used for transformation of most filamentous fungi and yeasts such as acetamidase genes or cDNAs (the amdS, niaD, facA genes or cDNAs from A. nidulans, A. oryzae, or A. niger), or genes providing resistance to antibiotics like G418, hygromycin, bleomycin, kanamycin, phleomycin or benomyl resistance (benA). Alternatively, specific selection markers can be used such as auxotrophic markers which require corresponding mutant host strains: e.g. URA3 (from S. cerevisiae or analogous genes from other yeasts), pyrG or pyrA (from A. nidulans or A. niger), argB (from A. nidulans or A. niger) or trpC. In a preferred embodiment the selection marker is deleted from the transformed host cell after introduction of the expression construct so as to obtain transformed host cells capable of producing the polypeptide which are free of selection marker genes.

Other markers include ATP synthetase subunit 9 (oliC), orotidine-5′-phosphate-decarboxylase (pvrA), the bacterial G418 resistance gene (useful in yeast, but not in filamentous fungi), the ampicillin resistance gene (E. coli), the neomycin resistance gene (Bacillus) and the E. coli uidA gene, coding for glucuronidase (GUS). Vectors may be used in vitro, for example for the production of RNA or to transfect or transform a host cell.

For most filamentous fungi and yeast, the expression construct is preferably integrated into the genome of the host cell in order to obtain stable transformants. However, for certain yeasts suitable episomal vector systems are also available into which the expression construct can be incorporated for stable and high level expression. Examples thereof include vectors derived from the 2 μm, CEN and pKD1 plasmids of Saccharomyces and Kluyveromyces, respectively, or vectors containing an AMA sequence (e.g. AMA1 from Aspergillus). When expression constructs are integrated into host cell genomes, the constructs are either integrated at random loci in the genome, or at predetermined target loci using homologous recombination, in which case the target loci preferably comprise a highly expressed gene. A highly expressed gene is a gene whose mRNA can make up at least 0.01% (w/w) of the total cellular mRNA, for example under induced conditions, or alternatively, a gene whose gene product can make up at least 0.2% (w/w) of the total cellular protein, or, in case of a secreted gene product, can be secreted to a level of at least 0.05 g/l.

An expression construct for a given host cell will usually contain the following elements operably linked to each other in consecutive order from the 5′-end to 3′-end relative to the coding strand of the sequence encoding the polypeptide of the first aspect: (1) a promoter sequence capable of directing transcription of the DNA sequence encoding the polypeptide in the given host cell, (2) preferably, a 5′-untranslated region (leader), (3) optionally, a signal sequence capable of directing secretion of the polypeptide from the given host cell into the culture medium, (4) the DNA sequence encoding a mature and preferably active form of the polypeptide, and preferably also (5) a transcription termination region (terminator) capable of terminating transcription downstream of the DNA sequence encoding the polypeptide.

Downstream of the DNA sequence encoding the polypeptide, the expression construct preferably contains a 3′ untranslated region containing one or more transcription termination sites, also referred to as a terminator. The origin of the terminator is less critical. The terminator can for example be native to the DNA sequence encoding the polypeptide. However, preferably a yeast terminator is used in yeast host cells and a filamentous fungal terminator is used in filamentous fungal host cells. More preferably, the terminator is endogenous to the host cell in which the DNA sequence encoding the polypeptide is expressed.

Enhanced expression of the polynucleotide encoding the polypeptide of the invention may also be achieved by the selection of heterologous regulatory regions, e.g. promoter, signal sequence and terminator regions, which serve to increase expression and, if desired, secretion levels of the protein of interest from the chosen expression host and/or to provide for the inducible control of the expression of the polypeptide of the invention.

Aside from the promoter native to the gene encoding the polypeptide of the invention, other promoters may be used to direct expression of the polypeptide of the invention. The promoter may be selected for its efficiency in directing the expression of the polypeptide of the invention in the desired expression host.

Promoters/enhancers and other expression regulation signals may be selected to be compatible with the host cell for which the expression vector is designed. For example prokaryotic promoters may be used, in particular those suitable for use in E. coli strains. When expression of the polypeptides of the invention is carried out in mammalian cells, mammalian promoters may be used. Tissues-specific promoters, for example hepatocyte cell-specific promoters, may also be used. Viral promoters may also be used, for example the Moloney murine leukaemia virus long terminal repeat (MMLV LTR), the rous sarcoma virus (RSV) LTR promoter, the SV40 promoter, the human cytomegalovirus (CMV) IE promoter, herpes simplex virus promoters or adenovirus promoters.

Suitable yeast promoters include the S. cerevisiae GAL4 and ADH promoters and the S. pombe nmt1 and adh promoter. Mammalian promoters include the metallothionein promoter which can be induced in response to heavy metals such as cadmium. Viral promoters such as the SV40 large T antigen promoter or adenovirus promoters may also be used. All these promoters are readily available in the art.

Mammalian promoters, such as β-actin promoters, may be used. Tissue-specific promoters, in particular endothelial or neuronal cell specific promoters (for example the DDAHI and DDAHII promoters), are especially preferred. Viral promoters may also be used, for example the Moloney murine leukaemia virus long terminal repeat (MMLV LTR), the rous sarcoma virus (RSV) LTR promoter, the SV40 promoter, the human cytomegalovirus (CMV) IE promoter, adenovirus, HSV promoters (such as the HSV IE promoters), or HPV promoters, particularly the HPV upstream regulatory region (URR). Viral promoters are readily available in the art.

A variety of promoters can be used that are capable of directing transcription in the host cells of the invention. Preferably the promoter sequence is derived from a highly expressed gene as previously defined. Examples of preferred highly expressed genes from which promoters are preferably derived and/or which are comprised in preferred predetermined target loci for integration of expression constructs, include but are not limited to genes encoding glycolytic enzymes such as triose-phosphate isomerases (TPI), glyceraldehyde-phosphate dehydrogenases (GAPDH), phosphoglycerate kinases (PGK), pyruvate kinases (PYK), alcohol dehydrogenases (ADH), as well as genes encoding amylases, glucoamylases, proteases, xylanases, cellobiohydrolases, β-galactosidases, alcohol (methanol) oxidases, elongation factors and ribosomal proteins. Specific examples of suitable highly expressed genes include e.g. the LAC4 gene from Kluyveromyces sp., the methanol oxidase genes (AOX and MOX) from Hansenula and Pichia, respectively, the glucoamylase (glaA) genes from A. niger and A. awamori, the A. oryzae TAKA-amylase gene, the A. nidulans gpdA gene and the T. reesei cellobiohydrolase genes.

Examples of strong constitutive and/or inducible promoters which are preferred for use in fungal expression hosts are those which are obtainable from the fungal genes for xylanase (xlnA), phytase, ATP-synthetase subunit 9 (oliC), triose phosphate isomerase (tpi), alcohol dehydrogenase (AdhA), amylase (amy), amyloglucosidase (AG—from the glaA gene), acetamidase (amdS) and glyceraldehyde-3-phosphate dehydrogenase (gpd) promoters.

Examples of strong yeast promoters which may be used include those obtainable from the genes for alcohol dehydrogenase, lactase, 3-phosphoglycerate kinase and triosephosphate isomerase.

Examples of strong bacterial promoters which may be used include the amylase and SPo2 promoters as well as promoters from extracellular protease genes.

Promoters suitable for plant cells which may be used include napaline synthase (nos), octopine synthase (ocs), mannopine synthase (mas), ribulose small subunit (rubisco ssu), histone, rice actin, phaseolin, cauliflower mosaic virus (CMV) 35S and 19S and circovirus promoters.

The vector may further include sequences flanking the polynucleotide giving rise to RNA which comprise sequences homologous to ones from eukaryotic genomic sequences, preferably mammalian genomic sequences, or viral genomic sequences. This will allow the introduction of the polynucleotides of the invention into the genome of eukaryotic cells or viruses by homologous recombination. In particular, a plasmid vector comprising the expression cassette flanked by viral sequences can be used to prepare a viral vector suitable for delivering the polynucleotides of the invention to a mammalian cell. Other examples of suitable viral vectors include herpes simplex viral vectors and retroviruses, including lentiviruses, adenoviruses, adeno-associated viruses and HPV viruses (such as HPV-16 or HPV-18). Gene transfer techniques using these viruses are known to those skilled in the art. Retrovirus vectors for example may be used to stably integrate the polynucleotide giving rise to the antisense RNA into the host genome. Replication-defective adenovirus vectors by contrast remain episomal and therefore allow transient expression.

The vector may contain a polynucleotide of the invention oriented in an antisense direction to provide for the production of antisense RNA. This may be used to reduce, if desirable, the levels of expression of the polypeptide.

### Host Cells and Expression

In a further aspect the invention provides a process for preparing a polypeptide of the invention which comprises cultivating a host cell transformed or transfected with an expression vector as described above under conditions suitable for expression by the vector of a coding sequence encoding the polypeptide, and recovering the expressed polypeptide. Polynucleotides of the invention can be incorporated into a recombinant replicable vector, such as an expression vector. The vector may be used to replicate the nucleic acid in a compatible host cell. Thus in a further embodiment, the invention provides a method of making a polynucleotide of the invention by introducing a polynucleotide of the invention into a replicable vector, introducing the vector into a compatible host cell, and growing the host cell under conditions which bring about the replication of the vector. The vector may be recovered from the host cell. Suitable host cells include bacteria such as E. coli, yeast, mammalian cell lines and other eukaryotic cell lines, for example insect cells such as Sf9 cells and (e.g. filamentous) fungal cells.

Preferably the polypeptide is produced as a secreted protein in which case the DNA sequence encoding a mature form of the polypeptide in the expression construct is operably linked to a DNA sequence encoding a signal sequence. In the case where the gene encoding the secreted protein has in the wild type strain a signal sequence preferably the signal sequence used will be native (homologous) to the DNA sequence encoding the polypeptide. Alternatively the signal sequence is foreign (heterologous) to the DNA sequence encoding the polypeptide, in which case the signal sequence is preferably endogenous to the host cell in which the DNA sequence is expressed. Examples of suitable signal sequences for yeast host cells are the signal sequences derived from yeast MFalpha genes. Similarly, a suitable signal sequence for filamentous fungal host cells is e.g. a signal sequence derived from a filamentous fungal amyloglucosidase (AG) gene, e.g. the A. niger glaA gene. This signal sequence may be used in combination with the amyloglucosidase (also called (gluco)amylase) promoter itself, as well as in combination with other promoters. Hybrid signal sequences may also be used within the context of the present invention.

Preferred heterologous secretion leader sequences are those originating from the fungal amyloglucosidase (AG) gene (glaA—both 18 and 24 amino acid versions e.g. from Aspergillus), the MFalpha gene (yeasts e.g. Saccharomyces and Kluyveromyces) or the alpha-amylase gene (Bacillus).

The vectors may be transformed or transfected into a suitable host cell as described above to provide for expression of a polypeptide of the invention. This process may comprise culturing a host cell transformed with an expression vector as described above under conditions suitable for expression of the polypeptide, and optionally recovering the expressed polypeptide.

A further aspect of the invention thus provides host cells transformed or transfected with or comprising a polynucleotide or vector of the invention. Preferably the polynucleotide is carried in a vector which allows the replication and expression of the polynucleotide. The cells will be chosen to be compatible with the said vector and may for example be prokaryotic (for example bacterial), or eukaryotic fungal, yeast or plant cells.

The invention encompasses processes for the production of a polypeptide of the invention by means of recombinant expression of a DNA sequence encoding the polypeptide. For this purpose the DNA sequence of the invention can be used for gene amplification and/or exchange of expression signals, such as promoters, secretion signal sequences, in order to allow economic production of the polypeptide in a suitable homologous or heterologous host cell. A homologous host cell is herein defined as a host cell which is of the same species or which is a variant within the same species as the species from which the DNA sequence is derived.

Suitable host cells are preferably prokaryotic microorganisms such as bacteria, or more preferably eukaryotic organisms, for example fungi, such as yeasts or filamentous fungi, or plant cells. In general, yeast cells are preferred over filamentous fungal cells because they are easier to manipulate. However, some proteins are either poorly secreted from yeasts, or in some cases are not processed properly (e.g. hyperglycosylation in yeast). In these instances, a filamentous fungal host organism should be selected.

Bacteria from the genus Bacillus are very suitable as heterologous hosts because of their capability to secrete proteins into the culture medium. Other bacteria suitable as hosts are those from the genera Streptomyces and Pseudomonas. A preferred yeast host cell for the expression of the DNA sequence encoding the polypeptide is one of the genus Saccharomyces, Kluyveromyces, Hansenula, Pichia, Yarrowia, or Schizosaccharomyces. More preferably, a yeast host cell is selected from the group consisting of the species Saccharomyces cerevisiae, Kluyveromyces lactis (also known as Kluyveromyces marxianus var. lactis), Hansenula polymorpha, Pichia pastoris, Yarrowia lipolytica, and Schizosaccharomyces pombe.

Most preferred for the expression of the DNA sequence encoding the polypeptide are, however, filamentous fungal host cells. Preferred filamentous fungal host cells are selected from the group consisting of the genera Aspergillus, Trichoderma, Fusarium, Disporotrichum, Penicillium, Acremonium, Neurospora, Thermoascus, Myceliophtora, Sporotrichum, Thielavia, and Talaromyces. More preferably a filamentous fungal host cell is of the species Aspergillus oryzae, Aspergillus sojae or Aspergillus nidulans or is of a species from the Aspergillus niger Group (as defined by Raper and Fennell, The Genus Aspergillus, The Williams & Wilkins Company, Baltimore, pp 293-344, 1965). These include but are not limited to Aspergillus niger, Aspergillus awamori, Aspergillus tubigensis, Aspergillus aculeatus, Aspergillus foetidus, Aspergillus nidulans, Aspergillus japonicus, Aspergillus otyzae and Aspergillus ficuum, and also those of the species Trichoderma reesei, Fusarium graminearum, Penicillium chrysogenum, Acremonium alabamense, Neurospora crassa, Myceliophtora thermophilum, Sporotrichum cellulophilum, Disporotrichum dimorphosphorum and Thielavia terrestris.

Examples of preferred expression hosts within the scope of the present invention are fungi such as Aspergillus species (in particular those described in EP-A-184,438 and EP-A-284,603) and Trichoderma species; bacteria such as Bacillus species (in particular those described in EP-A-134,048 and EP-A-253,455), especially Bacillus subtilis, Bacillus licheniformis, Bacillus amyloliquefaciens, Pseudomonas species; and yeasts such as Kluyveromyces species (in particular those described in EP-A-096,430 such as Kluyveromyces lactis and in EP-A-301,670) and Saccharomyces species, such as Saccharomyces cerevisiae.

Host cells according to the invention include plant cells, and the invention therefore extends to transgenic organisms, such as plants and parts thereof, which contain one or more cells of the invention. The cells may heterologously express the polypeptide of the invention or may heterologously contain one or more of the polynucleotides of the invention. The transgenic (or genetically modified) plant may therefore have inserted (typically stably) into its genome a sequence encoding the polypeptides of the invention. The transformation of plant cells can be performed using known techniques, for example using a Ti or a Ri plasmid from Agrobacterium tumefaciens. The plasmid (or vector) may thus contain sequences necessary to infect a plant, and derivatives of the Ti and/or Ri plasmids may be employed.

The host cell may overexpress the polypeptide, and techniques for engineering over-expression are well known and can be used in the present invention. The host may thus have two or more copies of the polynucleotide.

Alternatively, direct infection of a part of a plant, such as a leaf, root or stem can be effected. In this technique the plant to be infected can be wounded, for example by cutting the plant with a razor, puncturing the plant with a needle or rubbing the plant with an abrasive. The wound is then innoculated with the Agrobacterium. The plant or plant part can then be grown on a suitable culture medium and allowed to develop into a mature plant. Regeneration of transformed cells into genetically modified plants can be achieved by using known techniques, for example by selecting transformed shoots using an antibiotic and by sub-culturing the shoots on a medium containing the appropriate nutrients, plant hormones and the like.

### Culture of Host Cells and Recombinant Production

The invention also includes cells that have been modified to express the glucotransferase or a variant thereof. Such cells include transient, or preferably stably modified higher eukaryotic cell lines, such as mammalian cells or insect cells, lower eukaryotic cells, such as yeast and filamentous fungal cells or prokaryotic cells such as bacterial cells.

It is also possible for the polypeptides of the invention to be transiently expressed in a cell line or on a membrane, such as for example in a baculovirus expression system. Such systems, which are adapted to express the proteins according to the invention, are also included within the scope of the present invention.

According to the present invention, the production of the polypeptide of the invention can be effected by the culturing of microbial expression hosts, which have been transformed with one or more polynucleotides of the present invention, in a conventional nutrient fermentation medium.

The recombinant host cells according to the invention may be cultured using procedures known in the art. For each combination of a promoter and a host cell, culture conditions are available which are conducive to the expression the DNA sequence encoding the polypeptide. After reaching the desired cell density or titre of the polypeptide the culturing is ceased and the polypeptide is recovered using known procedures.

The fermentation medium can comprise a known culture medium containing a carbon source (e.g. glucose, maltose, molasses, etc.), a nitrogen source (e.g. ammonium sulphate, ammonium nitrate, ammonium chloride, etc.), an organic nitrogen source (e.g. yeast extract, malt extract, peptone, etc.) and inorganic nutrient sources (e.g. phosphate, magnesium, potassium, zinc, iron, etc.). Optionally, an inducer (dependent on the expression construct used) may be included or subsequently be added.

The selection of the appropriate medium may be based on the choice of expression host and/or based on the regulatory requirements of the expression construct. Suitable media are well-known to those skilled in the art. The medium may, if desired, contain additional components favoring the transformed expression hosts over other potentially contaminating microorganisms.

The fermentation may be performed over a period of from 0.5-30 days. Fermentation may be a batch, continuous or fed-batch process, at a suitable temperature in the range of between 0° C. and 45° C. and, for example, at a pH from 2 to 10. Preferred fermentation conditions include a temperature in the range of between 20° C. and 37° C. and/or a pH between 3 and 9. The appropriate conditions are usually selected based on the choice of the expression host and the protein to be expressed.

After fermentation, if necessary, the cells can be removed from the fermentation broth by means of centrifugation or filtration. After fermentation has stopped or after removal of the cells, the polypeptide of the invention may then be recovered and, if desired, purified and isolated by conventional means. The glucotransferase of the invention can be purified from fungal mycelium or from the culture broth into which the glucotransferase is released by the cultured fungal cells.

In a preferred embodiment the polypeptide is obtained from a fungus, more preferably from an Aspergillus, most preferably from Aspergillus niger.

### Modifications

Polypeptides of the invention may be chemically modified, e.g. post-translationally modified. For example, they may be glycosylated (one or more times) or comprise modified amino acid residues. They may also be modified by the addition of histidine residues to assist their purification or by the addition of a signal sequence to promote secretion from the cell. The polypeptide may have amino- or carboxyl-terminal extensions, such as an amino-terminal methionine residue, a small linker peptide of up to about 20-25 residues, or a small extension that facilitates purification, such as a poly-histidine tract, an antigenic epitope or a binding domain.

A polypeptide of the invention may be labelled with a revealing label. The revealing label may be any suitable label which allows the polypeptide to be detected. Suitable labels include radioisotopes, e.g. ¹²⁵I, ³⁵S, enzymes, antibodies, polynucleotides and linkers such as biotin.

The polypeptides may be modified to include non-naturally occurring amino acids or to increase the stability of the polypeptide. When the proteins or peptides are produced by synthetic means, such amino acids may be introduced during production. The proteins or peptides may also be modified following either synthetic or recombinant production.

The polypeptides of the invention may also be produced using D-amino acids. In such cases the amino acids will be linked in reverse sequence in the C to N orientation. This is conventional in the art for producing such proteins or peptides.

A number of side chain modifications are known in the art and may be made to the side chains of the proteins or peptides of the present invention. Such modifications include, for example, modifications of amino acids by reductive alkylation by reaction with an aldehyde followed by reduction with NaBH₄, amidination with methylacetimidate or acylation with acetic anhydride.

The sequences provided by the present invention may also be used as starting materials for the construction of “second generation” enzymes. “Second generation” glucotransferases are glucotransferases, altered by mutagenesis techniques (e.g. site-directed mutagenesis), which have properties that differ from those of wild-type glucotransferase or recombinant glucotransferases such as those produced by the present invention. For example, their temperature or pH optimum, specific activity, substrate affinity or thermostability may be altered so as to be better suited for use in a particular process.

Amino acids essential to the activity of the glucotransferase of the invention, and therefore preferably subject to substitution, may be identified according to procedures known in the art, such as site-directed mutagenesis or alanine-scanning mutagenesis. In the latter technique mutations are introduced at every residue in the molecule, and the resultant mutant molecules are tested for biological activity (e.g. glucotransferase activity) to identify amino acid residues that are critical to the activity of the molecule. Sites of enzyme-substrate interaction can also be determined by analysis of crystal structure as determined by such techniques as nuclear magnetic resonance, crystallography or photo-affinity labelling.

The use of yeast and filamentous fungal host cells is expected to provide for such post-translational modifications (e.g. proteolytic processing, myristilation, glycosylation, truncation, and tyrosine, serine or threonine phosphorylation) as may be needed to confer optimal biological activity on recombinant expression products of the invention.

### Preparations

Polypeptides of the invention may be in an isolated form. It will be understood that the polypeptide may be mixed with carriers or diluents which will not interfere with the intended purpose of the polypeptide and still be regarded as isolated. A polypeptide of the invention may also be in a substantially purified form, in which case it will generally comprise the polypeptide in a preparation in which more than 70%, e.g. more than 80%, 90%, 95%, 98% or 99% of the proteins in the preparation is a polypeptide of the invention.

Polypeptides of the invention may be provided in a form such that they are outside their natural cellular environment. Thus, they may be substantially isolated or purified, as discussed above, or in a cell in which they do not occur in nature, for example a cell of other fungal species, animals, plants or bacteria.

### Removal or Reduction of Glucotransferase Activity

The present invention also relates to methods for producing a mutant cell of a parent cell, which comprises disrupting or deleting the endogenous nucleic acid sequence encoding the polypeptide or a control sequence thereof, which results in the mutant cell producing less of the polypeptide than the parent cell.

The construction of strains which have reduced glucotransferase activity may be conveniently accomplished by modification or inactivation of a nucleic acid sequence necessary for expression of the glucotransferase in the cell. The nucleic acid sequence to be modified or inactivated may be, for example, a nucleic acid sequence encoding the polypeptide or a part thereof essential for exhibiting glucotransferase activity, or the nucleic acid sequence may have a regulatory function required for the expression of the polypeptide from the coding sequence of the nucleic acid sequence. An example of such a regulatory or control sequence may be a promoter sequence or a functional part thereof, i.e., a part which is sufficient for affecting expression of the polypeptide. Other control sequences for possible modification include, but are not limited to, a leader sequence, a polyadenylation sequence, a propeptide sequence, a signal sequence, and a termination sequence.

Modification or inactivation of the nucleic acid sequence may be performed by subjecting the cell to mutagenesis and selecting cells in which the glucotransferase producing capability has been reduced or eliminated. The mutagenesis, which may be specific or random, may be performed, for example, by use of a suitable physical or chemical mutagenizing agent, by use of a suitable oligonucleotide, or by subjecting the DNA sequence to PCR mutagenesis. Furthermore, the mutagenesis may be performed by use of any combination of these mutagenizing agents.

Examples of a physical or chemical mutagenizing agent suitable for the present purpose include ultraviolet (UV) irradiation, hydroxylamine, N-methyl-N′-nitro-N-nitrosoguanidine (MNNG), O-methyl hydroxylamine, nitrous acid, ethyl methane sulphonate (EMS), sodium bisulphite, formic acid, and nucleotide analogues. When such agents are used, the mutagenesis is typically performed by incubating the cell to be mutagenized in the presence of the mutagenizing agent of choice under suitable conditions, and selecting for cells exhibiting reduced or no expression of glucotransferase activity.

Modification or inactivation of production of a polypeptide of the present invention may be accomplished by introduction, substitution, or removal of one or more nucleotides in the nucleic acid sequence encoding the polypeptide or a regulatory element required for the transcription or translation thereof. For example, nucleotides may be inserted or removed so as to result in the introduction of a stop codon, the removal of the start codon, or a change of the open reading frame. Such modification or inactivation may be accomplished by site-directed mutagenesis or PCR mutagenesis in accordance with methods known in the art.

Although, in principle, the modification may be performed in vivo, i.e., directly on the cell expressing the nucleic acid sequence to be modified, it is preferred that the modification be performed in vitro as exemplified below.

An example of a convenient way to inactivate or reduce production of the glucotransferase by a host cell of choice is based on techniques of gene replacement or gene interruption. For example, in the gene interruption method, a nucleic acid sequence corresponding to the endogenous gene or gene fragment of interest is mutagenized in vitro to produce a defective nucleic acid sequence which is then transformed into the host cell to produce a defective gene. By homologous recombination, the defective nucleic acid sequence replaces the endogenous gene or gene fragment. Preferably the defective gene or gene fragment also encodes a marker which may be used to select for transformants in which the gene encoding the polypeptide has been modified or destroyed.

Alternatively, modification or inactivation of the nucleic acid sequence encoding a polypeptide of the present invention may be achieved by established anti-sense techniques using a nucleotide sequence complementary to the polypeptide encoding sequence. More specifically, production of the polypeptide by a cell may be reduced or eliminated by introducing a nucleotide sequence complementary to the nucleic acid sequence encoding the polypeptide. The antisense polynucleotide will then typically be transcribed in the cell and will be capable of hybridizing to the mRNA encoding the glucotransferase. Under conditions allowing the complementary antisense nucleotide sequence to hybridize to the mRNA, the amount of the glucotransferase produced in the cell will be reduced or eliminated.

It is preferred that the cell to be modified in accordance with the methods of the present invention is of microbial origin, for example, a fungal strain which is suitable for the production of desired protein products, either homologous or heterologous to the cell. The present invention further relates to a mutant cell of a parent cell which comprises a disruption or deletion of the endogenous nucleic acid sequence encoding the polypeptide or a control sequence thereof, which results in the mutant cell producing less of the polypeptide than the parent cell.

The polypeptide-deficient mutant cells so created are particularly useful as host cells for the expression of homologous and/or heterologous polypeptides. Therefore, the present invention further relates to methods for producing a homologous or heterologous polypeptide comprising (a) culturing the mutant cell under conditions conducive for production of the polypeptide; and (b) recovering the polypeptide. In the present context, the term “heterologous polypeptides” is defined herein as polypeptides which are not native to the host cell, a native protein in which modifications have been made to alter the native sequence, or a native protein whose expression is quantitatively altered as a result of a manipulation of the host cell by recombinant DNA techniques.

In a still further aspect, the present invention provides a method for producing a protein product essentially free of glucotransferase activity by fermentation of a cell which produces both an glucotransferase polypeptide of the present invention as well as the protein product of interest. The method comprises adding an effective amount of an agent capable of inhibiting glucotransferase activity to the fermentation broth either during or after the fermentation has been completed, recovering the product of interest from the fermentation broth, and optionally subjecting the recovered product to further purification. Alternatively, after cultivation the resultant culture broth can be subjected to a pH or temperature treatment so as to reduce the glucotransferase activity substantially, and allow recovery of the product from the culture broth. The combined pH or temperature treatment may be performed on an protein preparation recovered from the culture broth.

The methods of the present invention for producing an essentially glucotransferase-free product is of particular interest in the production of eukaryotic polypeptides, in particular in the production of fungal proteins such as enzymes. The glucotransferase-deficient cells may also be used to express heterologous proteins of interest for the food industry, or of pharmaceutical interest.

Preferred sources for the glucotransferase are obtained by cloning a microbial gene encoding a glucotransferase into a microbial host organism. More preferred sources for the glucotransferase are obtained by cloning an Aspergillus-derived gene encoding a glucotransferase into a host belonging to the genus of Aspergillus capable of overexpressing the glucotransferase gene.

### Table 1

Table 1 Alignment of the generally conserved regions of the α-amylase family as present in A. niger acid amylase and AmyA compared to homologous regions in AgtA, AgtB and AgtC. Catalytic residues are underlined, generally conserved residues are indicated in bold.

| Enzyme | Region I | Region II | Region III | Region IV |
|---|---|---|---|---|
| Acid amylase | LMVDVVPNH | DGLRIDSVLE | YCVGEVDN | NFIENHD |
| AmyA | LMVDVVANH | DGLRIDTVKH | YCIGEVLD | TFVENHD |
| AgtA | LMMDTVINN | DGLRIDAAKH | FMTGEVLQ | SFSENHD |
| AgtB | LLLDVVINN | DGLRIDAAKS | FMTGEVMD | NFIEDQD |
| AgtC | LMLDIVVGD | DGLRIDSVLN | FTVGEGAT | TFTANQD |
| Amino acid | 135-143 | 222-231 | 247-254 | 312-318 |
| numbering | | | | |
| according to | | | | |
| A. niger acid | | | | |
| amylase | | | | |

### LEGENDS TO THE FIGURES

FIG. 1

TLC analysis of the reaction products of AgtA (left side) and AgtB (right side) incubated with different substrates. 0.4 μg purified enzyme was incubated with 20 mM substrate for 1 h at 37° C. Marker containing maltooligosaccharides ranging from glucose to maltoheptaose (indicated M). Reaction products of AgtA/AgtB incubated with: maltotriose (lane 1/5), maltotetraose (lane 2/6), maltopentaose (lane 3/7), maltohexaose (lane 4/8).

FIG. 2

HPLC analysis of the reaction products formed by incubation of AgtA and AgtB on maltoheptaose. 0.4 μg purified enzyme was incubated with 20 mM substrate at 37° C., and samples were taken after 1 or 18 h of incubation.

(2A) Elution profile of a standard mixture containing glucose to maltoheptaose.

(2B) Reaction products of AgtA incubated for 1 h or 18 h on maltoheptaose, analysed by HPLC. Mainly maltooligosaccharides and some glucose are formed. The largest detectable oligosaccharide would fit DP 25.

(2C) Reaction products of AgtB incubated for 1 h or 18 h on maltoheptaose, analysed by HPLC. Products formed other than maltooligosaccharides are indicated with grey arrows. The largest detectable oligosaccharide would fit DP 30.

FIG. 3

HPLC analysis of reaction products formed by AgtA and AgtB incubated for 18 h on maltoheptaose, with a focus on small oligosaccharides. Abbreviations: G: glucose, G2: maltose, G3: maltotriose, IG: isomaltose, IG3: isomaltotriose, IG4: isomaltotetraose, IG5: isomaltopentaose, IG6: isomaltohexaose, F: Fructose, Leu: Leucrose, Suc: sucrose, Pan: panose.

(3A) Elution profile of a standard mixture.

(3B) Products formed by AgtA

(3C) Products formed by AgtB

FIG. 4

HPLC analysis of the products formed by AgtA and AgtB upon incubation with Paselli starch and nigerotriose. Peaks not representing maltooligosaccharides are indicated with grey arrows.

(4A) Reaction products of AgtA

(4B) Reaction products of AgtB

### SEQUENCES

SEQ ID NO: 1 is a nucleic acid sequence encoding a polypeptide having 4-α-glucanotransferase activity.

SEQ ID NO: 2 is a nucleic acid sequence encoding a polypeptide having 4-α-glucanotransferase activity.

SEQ ID NO: 3 is an amino acid sequence of a polypeptide having 4-α-glucanotransferase activity.

SEQ ID NO: 4 is a nucleic acid sequence encoding a polypeptide having 4-α-glucanotransferase activity.

SEQ ID NO: 5 is a nucleic acid sequence encoding a polypeptide having 4-α-glucanotransferase activity.

SEQ ID NO: 6 is an amino acid sequence of a polypeptide having 4-α-glucanotransferase activity.

SEQ ID NO: 7 is a nucleic acid sequence encoding a polypeptide having 4-α-glucanotransferase activity.

SEQ ID NO: 8 is a nucleic acid sequence encoding a polypeptide having 4-α-glucanotransferase activity.

SEQ ID NO: 9 is an amino acid sequence of a polypeptide having 4-α-glucanotransferase activity.

### EXAMPLES

### Example 1

### Identification of AgtA, AgtB and AgtC

The full genome sequence of Aspergillus niger strain CBS 513.88 (available from DSM, The Netherlands) was used as the starting point. A Hidden Markov model (HMM) profile was built using the HMMER package (Durbin & Eddy (1998), Biological sequence analysis: probabilistic models of proteins and nucleic acids. Cambridge University Press) based on the sequences of known fungal α-amylases, which were retrieved from the CAZY website (http://afmb.cnrs-mrs.fr/CAZY/). The profile was used to screen the A niger CBS513.88 genomic database using the WISE 2 package (Birney et al (2004) Genome Res 14, 988-995). The presence of a signal peptidase cleavage site and a glycosylphosphatidylinositol (GPI)-attachment site were predicted by web-based search tools (http://www.cbs.dtu.dk/services/SignalP/ and http://mendel.imp.univie.ac.at/sat/gpi/gpi_server.html). Amino acid sequence alignments were made using ClustaIX (1.83) and Genedoc (version 2.6.002) and adjusted manually if necessary.

Three genes were identified in the genome of Aspergillus niger CBS5130.88 that belong to family GH13, contain a signal peptide and a GPI-anchor. These genes were coded AgtA, AgtB and AgtC with gene accession numbers An09g03100, An12g02460 and An15g07800, (gene codes refer to the A niger genome sequence, available via DSM, The Netherlands). All three proteins contain at least 5 out of 7 generally conserved residues typical of family GH13. Their genomic sequences, cDNA sequences and protein sequences are given as seq ID's 1-9. The amino acid sequences of AgtA, AgtB and AgtC show 54-56% homology to A. niger acid amylase (Boel et al (1990) Biochem 29, 6244-6249). AgtA and AgtB have a mutual homology of 73% while the homology to AgtC is 53%. All three proteins contain at least 5 out of the 7 generally conserved residues typical of family GH13 (Table 1). However, in all three protein sequences the highly conserved H is in position 143, which is part of conserved region I, is absent and instead an Asp or Asn is found (numbering used is according to acid alpha-amylase of A. niger).

### Example 2

### Cloning of AgtA and AgtB

All basic molecular techniques were performed according to standard procedures (Sambrook et al (1989) Molecular cloning: a laboratory manual. Cold Spring Harbor press, NY). E coli TOP10 (Invitrogen, Carlsbad, USA) or DH5α (Statagene, La Jolla, USA) were used for transformation and amplification of recombinant DNA. Primers were obtained from Eurogentec (Seraing, Belgium) or Biolegio (Nijmegen, The Netherlands). All steps during the construction of the expression vectors were checked by restriction analysis, and the final plasmids were checked by sequencing (GATC Biotech AG, Konstanz, Germany). Genomic DNA was isolated from A niger N402 and NRRL3122 as described (Kolar et al (1988) Gene 62, 127-134). All PCR reactions were performed with 2.5 units of Pwo DNA polymerase (Roche, Indianapolis, USA), 1× buffer and 1 mM of each dNTP in a total volume of 25 μl. A cDNA library was produced from A niger N402 grown on mineral medium containing starch as the sole carbon source. The overexpression vector for the transformation of A niger was provided by dr J Benen (Wageningen University, The Netherlands), and was produced as follows: Gene pgalI was cloned into pPROM-S (Benen et al (1999) Eur J Biochem 259, 577-585) using NsiI and KpnI restriction sites. A NotI site was generated immediately downstream of the stop codon of the pgalI gene by site directed mutagenesis. The gene encoding aceetamidase (AmdS, Kolar et al (1988) Gene 62, 127-134) was amplified by PCR with specific primers from plasmid p3SR2 (Wernars et al (1985) Curr Genet. 9, 361-368) and cloned in front of the pki-promoter region using XbaI restriction sites, resulting in vector pKI-AmdS-pgalI. The constructs for overexpression of AgtA and AgtB were produced as follows: the complete gene sequences of genes AgtA and AgtB were amplified with specific primers from genomic DNA isolated from A. niger NRRL3122 (van Dijck et al (2003) Regul. Toxicol. Pharmacol. 38, 27-35). The primers contained restriction sites for NsiI and NotI, which were used to clone the gene fragments into vector pKI-AmdS-pgalI, thereby replacing the pgalI gene. This resulted in the vectors pKI-AmdS-AgtA and pKI-AmdS-AgtB.

### Example 3

### Production of AgtA and AgtB

A niger strain M00029-ΔaamA (Weenink et al (2006) Appl Microbiol Biotechnol 69, 711-717) was used as a host for protein over expression. This strain, derived from strain MGG029 (prtT glaA::fleo^{r} pyrG) is deficient in the expression of several extracellular proteases. It also has no glucoamylase gene (GlaA) and acid amylase gene (aamA) resulting in very poor growth on starch. Aspergillus strains were grown in Aspergillus Minimal Medium (MM) or Complete Medium (CM) which is MM with addition of 0.1% casaminoacids and 0.5% yeast extract (Oxoid, Basingstoke, UK). Cultures meant for protein production were grown in CMS (CM supplemented with 3% (w/v) sucrose). Spores were obtained by growing A. niger on CM supplemented with 2% agar for 4 days and scraping off the spores with 0.9% (w/v) NaCl. Liquid cultures were inoculated with 10⁶ spores I⁻¹ medium, and subsequently grown at 30° C. while shaking at 280 r.p.m. Transformation of A. niger was performed as described (Punt et al (1992) Meth. Enzymol. 216, 447-457) using lysing enzymes (Sigma, Zwijndrecht, The Netherlands). Transformants were successively selected on MM with 10 mM acetamide or 10 mM acrylamide as sole nitrogen source (Kelly et al (1985) EMBO J. 4, 475-479), both supplemented with 15 mM CsCl to reduce background growth. The proteins AgtA and AgtB were overproduced in A. niger by transformation with the plasmids pKI-AmdS-AgtA and pKI-AmdS-AgtB. Protein production of several transformants was verified (SDS-PAGE), and the best producing transformants for AgtA and AgtB respectively were selected for further experiments. Those transformants were grown in CSM for 3 days at 30° C. and 200 r.p.m. Mycelium was removed from the culture medium by filtration over miracloth. The medium was concentrated over a Centriprep YM-50 membrane filter and the concentrated protein was taken up in 20 mM Tris-HCl buffer pH 8.

The proteins were applied to an anion exchange column (Resource Q, 1 ml, Amersham Biosciences, New Jersey USA), equilibrated in 20 mM Tris.HCl buffer pH8. Proteins were eluted with a linear NaCl gradient (0-1 M NaCl) at a flow rate of 1 ml/min. Both AgtA and AgtB were eluted as a single peak at a concentration of 150 mM NaCl. At each stage of the protein purification, the protein amount was measured using the Bradford method with reagent from Bio-Rad, and purity was checked using SDS-PAGE analysis and staining with Biosafe Coomassie (Bio-Rad, Hercules, U.S.A.). The proteins eluted at an apparent molecular weight of 85,000 daltons. The same protein band was absent in the untransformed strain.

### Example 4

### Activity Measurements

All oligosaccharides were obtained from Sigma, except nigerotriose which was purchased from Dextra laboratories (Reading, U.K.), nigerose which was a gift from Nihon Shokuhin Kako Co. Ltd. (Shizuoka, Japan) and α-(1,3)-glucan isolated from A. nidulans which was a gift from Dr. B. J. Zonneveld (Leiden University, Leiden, The Netherlands). As soluble starch, Paselli SA2 with an average degree of polymerization of 50 (AVEBE, Foxhol, The Netherlands), was used.

Hydrolyzing activity on Paselli SA2 starch was determined by the incubation of two duplicate reactions containing 20 μg ml⁻¹ enzyme and 4% Paselli SA2 in 25 mM NaAc, pH 5.5 containing 1 mM CaCl₂. Reactions were incubated at 37° C. and samples were taken after several time intervals up to 3 h for the following analyses: twenty times diluted samples were analysed in triplicate for detection of reducing ends using the Nelson-Somogyi assay adapted for use in micro titre plates (Green et al (1989) Anal Biochem 182, 179-199). Ten times diluted samples from the same assays were used for the detection of glucose by using the Glucose GOD-PAP assay (Roche, Mannheim, Germany) in duplicate. Appropriate calibration curves and negative controls were included for all assays and reactions.

To determine the optimum pH for activity of AgtA and AgtB, 0.5 μl purified enzyme was incubated with 20 mM maltopentaose (G5) in a 20 μl reaction volume at 37° C., for 1 h at 11 different pH values. The reaction was buffered either by 30 mM NaAc buffer with a pH ranging from 4.2 to 7.0, or K₂HPO₄/KH₂PO₄ buffer with pH 6.3 to 8.0. 2 μl of the reaction product was spotted on a Thin Layer Chromatography (TLC) plate (Silica gel 60 F₂₅₄, Merck, Darmstadt, Germany) and, after drying, the plate was run for 6 h in a small amount of running buffer (butanol/ethanol/mQ 5/5/3 (v/v/v)). After running the plate was dried and sprayed with 50% sulphuric acid in methanol and left to develop for 10 min at 110° C.

Standard assay conditions for all further enzymatic reactions were as follows: 0.4 μg of purified enzyme was incubated in 20 μl 25 mM NaAc buffer pH 5.5 containing 1 mM CaCl₂ and 0.01% NaAzide in the presence of 20 mM oligosaccharide substrate and/or 4% Paselli SA2 starch or other polysaccharide, except nigeran and α-(1,3)-glucan. Nigeran and α-(1,3)-glucan were dissolved in 1 M NaOH. The pH of a 1% solution of these polysaccharides was adjusted to pH 5.5 with HAc and used in the reaction mixture at a final concentration of 0.5% to prevent precipitation. All reactions were incubated at 37° C. Reaction products were detected either by TLC or HPLC (Dionex) analysis. For TLC, 2 μl reaction product was spotted on a silica plate which was handled as described above. For HPLC analysis, 5 μl of the reaction mixture was dissolved in 1.5 ml 90% dimethylsulfoxide. Separation of oligosaccharides was achieved as described (Kralj et al (2004) Microbiol 150, 2099-2112).

### Example 5

### Glucanotransferase Activity of AgtA and AgtB on Maltooligosaccharides

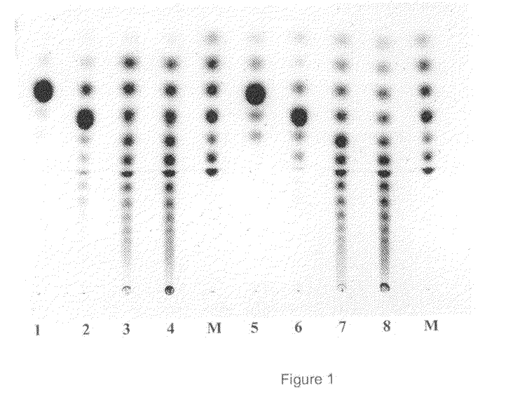

AgtA and AgtB were incubated in individual experiments with maltooligosaccharides ranging in size from maltose to maltohexaose and the products were analysed by TLC. Both proteins showed a clear glucanotransferase activity. When maltopentaose or maltohexaose were offered, both enzymes produced a range of oligosaccharides with reaction products of d. p. 15 or larger (FIG. 1). Maltose, maltotriose and maltotetraose were also converted by both enzymes but only to a very limited extent (less than 10%, as judged by spot intensities in FIG. 1). A small amount (less than 5% of total sugars) of glucose was formed by AgtB on all substrates tested. AgtA, on the contrary, did not produce glucose in significant amounts under the reaction conditions used. When incubated with 4% soluble starch both enzymes produced only very small amounts of reducing ends (less then 3 mM h⁻¹ μg⁻¹). To obtain an indication of the pH optimum, both enzymes were incubated with maltopentaose at different pH values and the products formed were analysed by TLC. AgtA was active between pH 4.5 and pH 6. AgtB showed activity between pH 4 and pH 5.5. Glucose polymers with different glycosidic linkages, like dextran (α-(1,6)-glycosidic bonds), nigeran (alternating α-(1,3)/α-(1,4)-glycosidic bonds), A. nidulans α-(1,3) glucan, a Lactobacillus reuteri exopolysaccharide containing α-(1,3)- and α-(1,6)-glycosidic bonds, and cellulose (β-(1,4)-glycosidic bonds) were tested as possible substrates but none of these were substrates for the hydrolysis or transferase reaction of AgtA or AgtB.

### Example 6

### Transglycosylation Products of AgtA and AgtB

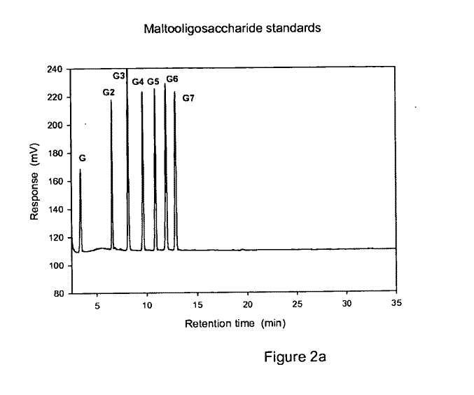

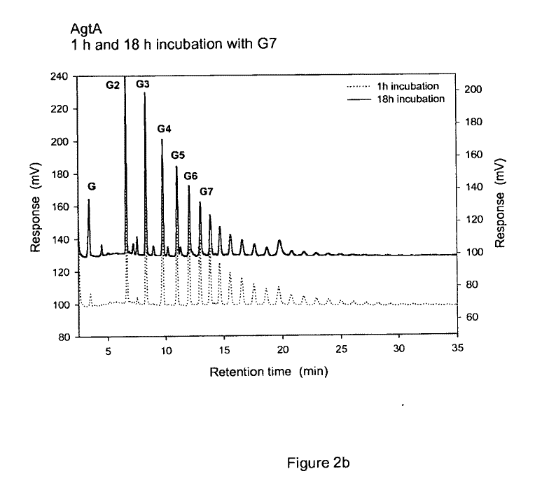

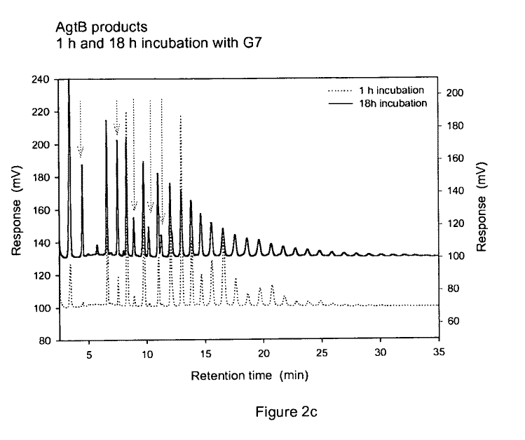

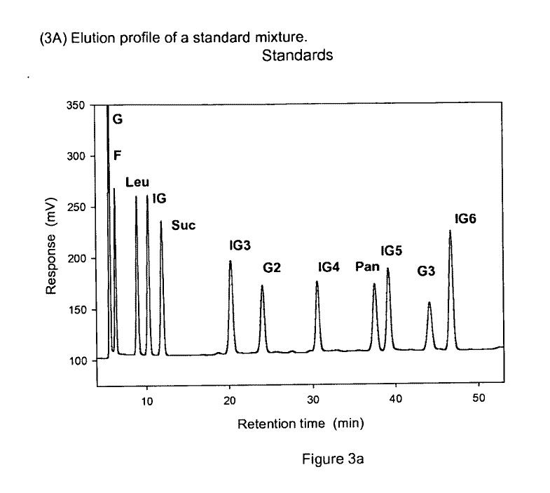

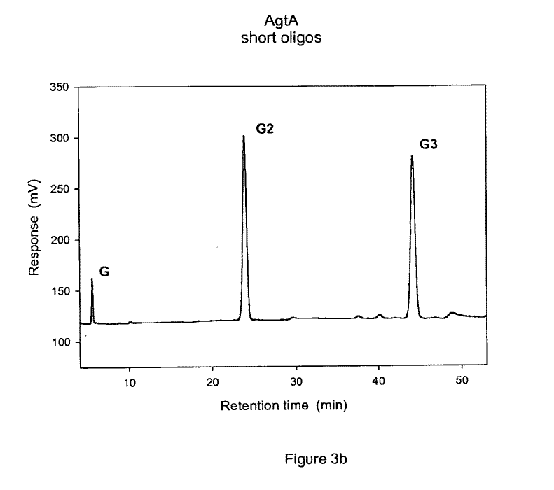

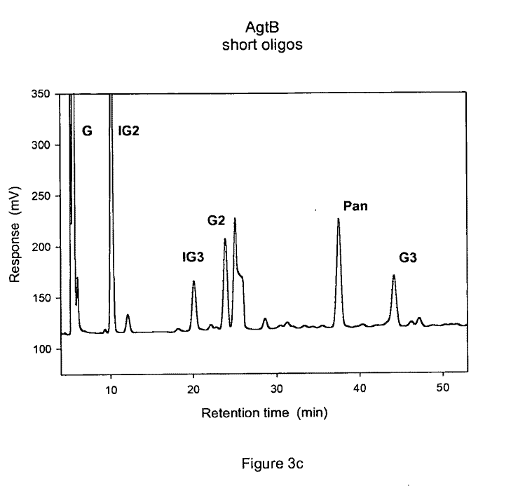

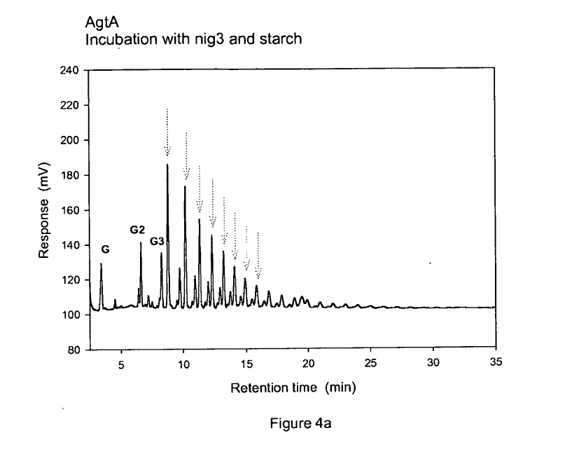

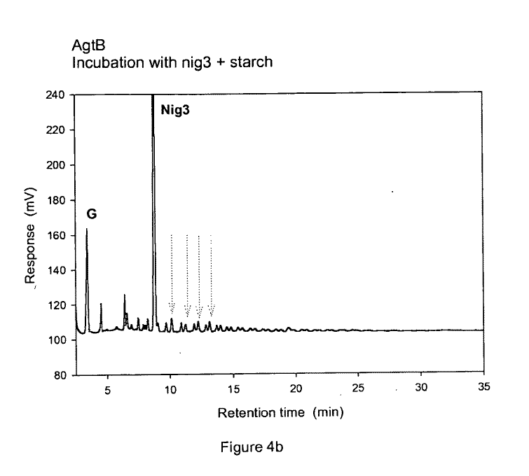

AgtA and AgtB were incubated under standard reaction conditions with maltopentaose (G5) or maltoheptaose (G7), and samples for HPLC analysis were taken after 1 and 18 h of incubation (FIGS. 2 B and C). AgtA produced virtually the same reaction products from G5 and G7. Oligosaccharides up to at least DP 21 and DP 17 (DP: degree of polymerisation) were formed after 1 and 18 h of incubation respectively with maltopentaose as a substrate. From maltoheptaose, oligosaccharides with respective lengths of at least DP 30 and DP 23 were formed after 1 and 18 h of incubation (FIG. 2B). The retention times of the major reaction products corresponded to those for standard maltooligosaccharide products.

Incubation of AgtB with maltopentaose and maltoheptaose also resulted in the clear formation of longer oligosaccharide products. From maltopentaose, products up to DP 15 and DP 21 were formed after 1 and 18 h respectively, while products made from maltoheptaose were even longer (DP 24 and DP 30 respectively) (FIG. 2C). Several products formed by AgtB had retention times that were clearly different from the retention times of the maltooligosaccharide standards (FIG. 2C). These products had the same retention times as the side products that were formed in very low amounts by AgtA. To identify these compounds, the products of the 18 h incubations of both AgtA and AgtB were analysed by HPLC with a focus on small oligosaccharides (FIG. 3). Isomaltose, isomaltotriose and panose (4-α-isomaltosylglucose) were identified as products of AgtB after 18 h incubation on both maltopentaose and maltoheptaose (FIG. 3C). At least two other products could not be identified due to a lack of suitable standards. Analysis of the products of AgtA confirmed the formation of maltooligosaccharides, while the amount of by products formed was too low for further identification (FIG. 3B).

### Example 7

### Use of α-(1,3)-glucoolgosaccharides as Acceptor Molecules

In the previous paragraph we showed that neither nigeran nor α-(1,3)-glucan were used as donor substrate by AgtA and AgtB. To investigate whether substrates with α-(1,3)-glycosidic bonds could be used as an acceptor substrate by AgtA or AgtB, both enzymes were incubated for 1 h with soluble starch and glucose, maltose, nigerose or nigerotriose (O-α-D-Glc-(1,3)-α-D-Glc-(1,3)-D-Glc) as acceptor substrates. Analysis of the reaction products by TLC revealed that glucose was not used as acceptor substrate by one of the enzymes but maltose was an efficient acceptor substrate. AgtA also formed a series of oligosaccharides using either nigerose or nigerotriose as acceptor substrates. The amount of products formed with nigerose or nigerotriose was lower than when maltose was used as a substrate. AgtB did not use nigerose or nigerotriose efficiently as acceptor substrate. No activity of AgtA or AgtB was observed on nigerose or nigerotriose as sole substrates. The use of nigerooligosaccharides as acceptor by AgtA was confirmed by HPLC analysis of the reaction products (FIG. 4A). The main products formed had slightly different retention times compared to the standard maltooligosaccharides probably caused by the incorporation of one or more α-(1,3)-glycosidic bonds. For AgtB a similar observation was done (FIG. 4B).

1. An isolated polypeptide which has 4-α-glucanotransferase activity and which has an amino acid sequence which has at least 60% amino acid sequence identity with amino acids 1 to 567, 1 to 549 or 1 to 555 of SEQ ID NO: 9, 6 or 3, respectively.

2. The polypeptide of claim 1 which has an amino acid sequence which has at least 50%, preferably at least 60%, preferably at least 65%, preferably at least 70%, more preferably at least 80%, even more preferably at least 90%, most preferably at least 95%, and even most preferably at least about 97% identity with amino acids 1 to 567, 1 to 549 or 1 to 555 of SEQ ID NO: 9, 6 or 3.

3. The polypeptide of claim 1, comprising the amino acid sequence of SEQ ID NO: 9, 6 or 3.

4. The polypeptide of claim 1, which is obtained from a fungus, preferably an Aspergillus, more preferably from Aspergillus niger.

5. An isolated polynucleotide comprising a nucleic acid sequence which encodes the polypeptide of claim 1.

6. A nucleic acid construct comprising the polynucleotide of claim 5 operably linked to one or more control sequences that direct the production of the polypeptide in a suitable expression host.

7. A recombinant expression vector comprising the nucleic acid construct of claim 6.

8. A recombinant host cell comprising the nucleic acid construct of claim 6 or a recombinant expression vector comprising said nucleic acid construct.

9. A method for producing the polypeptide of claim 1 comprising cultivating a host cell comprising a nucleic acid construct comprising a polynucleotide encoding the polypeptide under conditions suitable for production of the polypeptide; and recovering the polypeptide.

10. A method for producing the polypeptide according to claim 9 comprising cultivating a strain or said recombinant host cell, to produce a supernatant and/or cells comprising the polypeptide; and recovering the polypeptide.

11. A polypeptide produced by the method of claim 9.

12. A method for the transfer of a glucose moiety in a medium which comprises
(a) amylose containing starch or glucose-polymers containing α-(1,4) glycosidic bonds and consisting of at least five anhydroglucose units; and
(b) maltose, maltooligosaccharide, nigeran, nigerotriose or β-(1,3)-glucan, whereby a polypeptide having 4-α-glucanotransferase activity is used to transfer the is glucose moiety from amylose containing starch or glucose-polymers containing α-(1,4) glycosidic bonds and consisting of at least five anhydroglucose units to an acceptor molecule maltose, maltooligosaccharide, nigeran, nigerotriose or β-(1,3)-glucan using the polypeptide of claim 1.

13. A method according to claim 12 in which the transfer-percentage of sugars in aqueous solution to acceptor molecules other than water is higher than 90%, preferably higher than 95%.

14. A polypeptide having 4-α-glucanotransferase activity and which is able to use as donor substrates amylose containing starch and glucose-polymers containing α-(1,4) glycosidic bonds and consisting of at least five anhydroglucose units and as acceptor molecules maltose, maltooligosaccharides, nigeran, nigerotriose or β(1,3)-glucan.

15. A polypeptide according to claim 14 which has a transfer-percentage of sugars in aqueous solution to acceptor molecules other than water of higher than 90%, preferably higher than 95%.

16. Use of a polypeptide according to claim 1 in the preparation of food or feed.

17. Process for treating starch which comprises bringing the starch together with a polypeptide according to claim 1 for a period of time to treat the starch.

18. Process for producing a food or feed which comprises bringing starch together with a polypeptide according to claim 1 for a period of time to treat the starch and using this starch to produce the food or feed.

19. Process for producing an oligosaccharide which comprises bringing starch together for a period of time with a polypeptide according to claim 1 in the presence of a suitable acceptor substrate selected from the group consisting of maltose, maltoseoligosaccharides, nigeran, nigerotriose and β-(1,3)-glucan to form oligosaccharides.

20. Process for producing a food or feed which comprises bringing starch together for a period of time with a polypeptide according to claim 1 in the presence of a suitable acceptor substrate selected from the group consisting of maltose, maltoseoligosaccharides, nigeran, nigerotriose and α-(1,3)-glucan to form oligosaccharides and using these oligosaccharides to produce the food or feed.
